(19) United States
(12) Patent Application Publication
(10) Pub. No.: US 2002/0187931 A1
(43) Pub. Date: Dec. 12, 2002

(54) Modulating cell survival by modulating huntingtin function

(76) Inventors: Michael Hayden, Vancouver (CA); Abigail S. Hackam, Baltimore, MD (US); Blair R. Leavitt, Vancouver (CA)

Correspondence Address: GREENBERG-TRAURIG, 1750 TYSONS BOULEVARD, 12TH FLOOR, MCLEAN, VA, 22102, US

(21) Appl. No.: 10/009,478

(22) PCT Filed: Apr. 12, 2001
(86) PCT No.: PCT/CA01/00495

(30) Foreign Application Priority Data
Apr. 13, 2000 (CA) 2,305,088
Dec. 12, 2000 (CA) 2,326,543

(51) Int. Cl. A61K 48/00; C07K 14/435; A61K 38/17

(52) U.S. Cl. 514/12; 530/350; 514/44

(57) ABSTRACT

The present invention provides a means of modulating cell survival by modulating wild-type huntingtin protein function by administration of wild-type huntingtin protein, biologically active fragments thereof and/or antagonists of wild-type huntingtin. Accordingly, the present invention also provides biologically active fragments of wild-type huntingtin, antagonists of wild-type huntingtin and nucleic acid sequences encoding the biologically active fragments and peptide antagonists, and their use to modulate cell survival. In particular, the invention provides for means to activate or attenuate cell death within tissue, in order to facilitate the treatment of conditions where there is a dysregulation of cell death or cellular proliferation. Therapeutic application of this invention pertains to diseases and disorders including, but not limited to, Huntington disease, neurodegenerative diseases, stroke, and cancer.

### FIELD OF INVENTION

The invention pertains to the field of medicine, specifically to therapeutic or preventative compositions for use in the treatment of mammalian conditions characterised by a dysregulation of cell death or cellular proliferation.

### BACKGROUND OF THE INVENTION

Huntington disease is a neurodegenerative disease caused by mutation of the huntingtin protein (known as huntingtin or htt). The physiological role of the huntingtin protein is currently unknown. HD (Huntington's Disease) is a devastating neurological disease which usually presents in mid adult life, affects approximately 1 in 10,000 individuals (Hayden 1981), and results in psychiatric disturbance, involuntary movement disorder, and cognitive decline associated with inexorable progression to death, typically 17 years following onset.

A recent epidemiological study shows that there is a lower incidence of cancer among patients with HD which appears to be related to intrinsic biologic factors (Sorensen et al. (1999) Cancer 86, 1342-1346). A possible explanation for this may be that the mutant huntingtin protects against cancer by inducing or increasing the rate of naturally occurring apoptosis in preneoplastic cells. This further implicates that the induced or increased apoptosis, resulting from the presence of mutant huntingtin, is a factor in the generation of HD. This theory is further supported by a cell culture study which demonstrated that the expression of truncated N-terminal huntingtin containing the expanded polyglutamine caused a repeat length- and dose-dependent increase in the formation of aggregates and cell death (Martindale et al. (1998) Nat. Genet. 18,150-154; Wang et al. (1999) Neuroreport. 10, 2435-2438). The researchers observed that caspases were activated, and the death substrates of caspases, lamin B and inhibitor of caspase-activated DNase (ICAD), were cleaved in this cell death process. These findings suggest that huntingtin with the polyglutamine expansion was responsible for cell death induction and that this cell death is mediated by caspases.

Apoptosis is also called “programmed cell death” or “cell suicide”. (Krammer et al. (1991) “Apoptosis in the APO-1 System”, Apoptosis: The Molecular Basis of Cell Death, pp. 87-99 Cold Spring Harbor Laboratory Press). When the normal function of apoptosis goes awry, the cause or the result can be one of a number of diseases, including: cancer, viral infections, autoimmune disease/allergies, neurodegeneration or cardiovascular diseases. In HD, it is not known how the mutant gene that is widely expressed results in selective neuronal death. Further, sequence analysis has revealed no obvious homology to other known genes and no structural motifs or functional domains have been identified which clearly provide insights into its function. In particular, the question of how these widely expressed genes cause selective neuronal death remains unanswered. In addition, the role of wild-type huntingtin protein in the normal life cycle of cells, involving the apoptotic pathway and cellular proliferation, throughout the body of an organism, remains to be determined.

Mice heterozygous for targeted disruption of the HD gene express half the normal levels of wild-type htt, and have previously been shown to develop neuronal degeneration in the basal ganglia (Nasir et al. (1995) Cell 81, 811-823; O'Kusky et al. (1999) Brain Res. 818, 468-479. Wild-type htt has also recently been shown to protect cells from apoptotic stimuli in vitro (Rigamonti et al. (2000) J. Neurosci. 20, 3705-3713).

The present invention establishes a biological function for huntingtin protein and/or biologically active fragments thereof, in addition to nucleic acid sequences encoding such proteins.

### SUMMARY OF THE INVENTION

It is an object of this invention to provide a means of modulating cell survival by modulating the function of huntingtin protein, and/or biologically active fragments thereof. In particular, the invention provides a means to modulate the cellular balance between proliferation and death in order to facilitate the treatment of conditions where there is a dysregulation of apoptosis or cellular proliferation. Therapeutic application of this invention pertains to diseases and disorders characterised by an increase in apoptosis including, but not limited to, Huntington disease, neurodegenerative diseases and stroke, in addition to diseases characterised by dysregulated cellular proliferation, such as cancer.

According to one aspect of the present invention there is provided nucleic acids encoding huntingtin protein, or fragment thereof, and their use to modulate cell survival.

According to another aspect of the present invention there is provided a use of huntingtin protein, or biologically active fragments thereof that will increase the level of huntingtin protein function within cellular material, for the treatment of conditions characterised by dysregulated cell death.

According to another aspect of the present invention there is provided a use of antagonists to huntingtin protein to decrease huntingtin protein function in the treatment of conditions characterised by dysregulation of cellular proliferation.

According to one aspect of the present invention there is provided pharmaceutical compositions comprising huntingtin, a biologically active fragment of huntingtin, or a combination thereof and a pharmaceutically acceptable diluent or excipient.

According to another aspect of the present invention there is provided a use of huntingtin, a biologically active fragment of huntingtin, or a combination thereof for the preparation of a medicament for the treatment of conditions characterised by dysregulation of cell death or cell proliferation.

According to another aspect of the present invention there is provided compositions comprising a nucleic acid encoding huntingtin protein, a nucleic acid encoding an active fragment of huntingtin, or a combination thereof, and a pharmaceutically acceptable diluent or excipient, wherein said compositions are used for the treatment of conditions characterised by dysregulation of cell death and wherein said compositions attenuate cell death and/or increase cell proliferation.

According to another aspect of the present invention there is provided compositions comprising antagonists to huntingtin protein and a pharmaceutically acceptable diluent or excipient, wherein said compositions are used for the treatment of conditions characterised by dysregulation of cellular proliferation and wherein said compositions decrease cellular proliferation and/or activate apoptosis.

According to another aspect of the present invention there is provided an assay for screening for molecules having an anti-proliferative activity comprising the steps of transfecting NIH3T3 cells with huntingin, adding a candidate molecule to the transfected cells and comparing proliferation of the transfected cells treated with the candidate molecule with proliferation of the transfected cells in the absence of the candidate molecule, wherein an anti-proliferative effect is found where there is a decrease in proliferation of the treated and transfected cells in comparison to the untreated transfected cells.

### BRIEF DESCRIPTION OF THE FIGURES

FIG. 1 presents a schematic representation of the balance between normal cell proliferation and cell death. An imbalance will result in abnormal proliferation or abnormal cell death as is observed in various diseases and disorders.

FIG. 2 depicts the testicular morphology of yeast artificial chromosome (YAC) rescued huntingtin knockout mice. The YACs used to rescue mice contain the huntingtin gene with 18, 46 or 72 CAG repeats. The normal number of CAG repeats found in humans without HD is 18. Greater numbers of CAG repeats are associated with HD development, with 46 being an intermediate number and 72 being a high number of repeats. Semi-thin sections of testes stained with toluidine blue from 8 month old mice reveal the gross testicular morphology of mice with varying amounts of endogenous huntingtin rescued with YAC18 (a, b, c), YAC46 (d, e, f), and YAC72 (g, h, i) transgenes. Massive degeneration of spermatocytes is shown, and this novel cell death phenotype is CAG repeat size-dependent and is modulated by the level of endogenous huntingtin. The cell death is most pronounced in YAC72 rescued knockout mice, intermediate in YAC46 knockout mice and not present in YAC18 rescued mice.

FIG. 3 depicts protein aggregates in YAC72 rescued huntingtin knockout mice. Ultrastructural analysis of the testes of YAC72 mice lacking endogenous huntingtin revealed the presence of abnormal aggregates of intracellular protein (arrows) within spermatids, sertoli cells and sperm tails. The composition of these protein aggregates is currently unknown, but they resemble the ultrastructural appearance of huntingtin aggregates found in human HD brain tissue. Ectopic microtubule bundles and manchettes were also identified (arrows).

FIG. 4 depicts electron microscopic (EM) analysis of degenerating testicular cells from YAC72 rescued huntingtin knockout mice. Ultrastructural analysis of testes from YAC72 mice nullizygous for endogenous huntingtin reveals massive cell death of spermatids, phagocytosis of degenerating cells and formation of multinucleated giant cells.

FIG. 5 demonstrates that the C-terminus of huntingtin protein is anti-apoptotic. Using the methylthiazol tetrazolium (MTT) assay to measure cell viability, it is shown that expression of the C-terminus (C-ter) of huntingtin protects HEK 293T cells from tamoxifen induced cell death when compared with expression of β-galactosidase (LacZ) as a control.

FIG. 6 demonstrates that the C-terminus of huntingtin protein confers protection against huntingtin toxicity in NT2 cells. Mutant huntingtin protein (HD138) was co-transfected with control protein, pyruvate kinase (PK), or with huntingtin C-terminus (C-ter) and cell death in response to tamoxifen stimulus was measured. Expression of the C-terminus reduced HD138-dependent cell death (p=0.002).

FIG. 7 demonstrates that the C-terminus of huntingtin protein rescues HIP-1 toxicity in NT2 cells. Expression of the C-terminus in NT2 cells reduces HIP-1 mediated cell death, compared with expression of control protein (PK) (p<0.01).

FIG. 8 demonstrates that the C-terminus of huntingtin protein reduces mutant huntingtin protein aggregate formation. Expression of the C-terminus (C-ter) reduces the number of aggregates formed by a truncated version of mutant huntingtin protein (1955-128), compared with coexpression of huntingtin and the LacZ control. Aggregates were induced by tamoxifen stimulus (A) or by HIP-1 expression (B).

FIG. 9 shows results of studies with YAC transgenic mice expressing increased levels of wild-type human huntingtin are resistant to neurodegeneration following kainic acid-induced seizures. a, Quantification of degenerating hippocampal neurons following kainic acid-induced seizures in mice expressing 2-3 times the endogenous levels of wild-type huntingtin (212 line) and littermate controls (FVB/NJ). Average numbers of degenerating neurons per animal identified by Fluoro-Jade labeling are expressed for the CA1, CA3 and total hippocampal regions. Data is expressed as mean +/− SEM with significance determined using a two-tailed students t-test. Images of degenerating neurons (arrows) using Fluoro-Jade labeling (b. line 212 20x, c. FVB/NJ 20x, d. FVB/NJ 100x), silver staining (e. line 212 20x, f. FVB/NJ 20x, g. FVB/NJ 100x), and TUNEL staining (h. line 212 20x, i. FVB/NJ 20x,j. FVB/NJ 100x) within the hippocampus following KA-induced seizures.

FIG. 10 shows YAC transgenic mice expressing increased levels of wild-type human huntingtin have decreased caspase-3 activation following kainic acid-induced seizures. Quantification of hippocampal and cerebellar DEVD-ase activity following kainic acid-induced seizures in mice expressing 2-3 times the endogenous levels of wild-type huntingtin (212 line) and littermate controls (FVB/NJ). Data is expressed as mean +/− SEM with significance determined using a two-tailed students t-test.

FIG. 11 shows rescue of the Hdh nullizygous lethal phenotype by YAC transgenes expressing mutant huntingtin. Resultant genotypes for the F2 offspring of a cross between a YAC72 transgene positive, Hdh gene heterozygous mouse (+,+/− genotype) and a transgene negative, Hdh heterozygous mouse (−,+/− genotype). (a). The upper PCR bands represent the presence or absence of the YAC transgene and the lower bands represent the state of the endogenous Hdh gene. The mouse represented in lane two has the YAC72 transgene, but lacks the endogenous Hdh gene (+,−/− genotype). This mouse demonstrates that mutant human huntingtin expression form our YAC transgene rescued the Hdh nullizygous state. Mice with targeted disruption of the Hdh gene were rescued from the embryonic lethal phenotype equally by all three of the YAC transgenes described in this paper. The F2 offspring of our experimental breedings had the expected 1:2:1 ratio of genotypes for all of the YAC transgenes examined (B). Western blot analysis of huntingtin protein expression (C) confirmed the absence of endogenous htt protein in Hdh nullizygous mice (−/−) compared to wild-type (+/+) and demonstrated similar levels of human transgenic huntingtin expression in YAC18, YAC46, and YAC72 rescued Hdh nullizygous mice (+,−/−). Average testicular weight and epididymal sperm counts for YAC72+/+, YAC72+/−, and YAC72−/− mice at four months of age are shown in (D) and (E) respectively. YAC72−/− mice had significant testicular atrophy (p<10⁻⁵) and decreased sperm counts (p<10⁻⁵) compared to YAC72+/+ and YAC72+/− mice.

FIG. 12 shows testicular morphology of YAC transgene rescued Hdh nullizygous mice. Semi-thin sections of testes stained with toluidine blue from 8-month-old mice reveal the gross testicular morphology of mice with the YAC18 (a,b,c), YAC46 (d,e,f), and YAC72 (g,h,i) htt transgenes and either 100% of endogenous htt levels (+/+), 50% of endogenous htt levels (+/−) or absence of endogenous htt (−/−). Massive degeneration of spermatogenic cells occurs in the seminiferous tubules of mice expressing mutant htt with 46 or 72 polyglutamine repeats (panels i and f). The cell death is most pronounced in YAC72 (i), intermediate in YAC46 (f), and not present in YAC18 (c) rescued Hdh nullizygous mice. The human HD transgene in each of these lines of mice is identical except for the length of the CAG repeat, and these results suggest that this novel cell death phenotype is CAG repeat length-dependent. Increasing levels of endogenous htt markedly reduced the amount of spermatogenic cell degeneration (panels d,e and g,h) observed in YAC46 and YAC72 mice. (Bar=100 μm.)

FIG. 13 shows morphologic, biochemical, and ultrastructural evidence for apoptotic cell death in the testes of YAC72 mice lacking endogenous htt. Massive death of spermatogenic cells was observed in YAC72 mice lacking endogenous htt by toluidine blue staining (A) which revealed decreased numbers of spermatogenic cells, and a disordered epiythelium filled with vacuoles compared to the well large numbers of spermatocytes in well ordered stratified epithelium of YAC72+/+ mice (C). Increased apoptosis was evident in the testes of YAC72−/− mice by increased TUNEL labelling of spermatogenic cells (arrows in panel B) compared to YAC72 mice with normal levels of endogenous huntingtin (D). EM analysis of degenerating testicular cells from YAC72 (−/−) mice also provided evidence of apoptosis. Ultrastructural analysis of testes from YAC72 mice nullizygous for Hdh reveals massive cell death of spermatids, phagocytosis of degenerating cells, and formation of multinucleated giant cells. The epithelium of YAC72 mice tacking endogenous htt was characterized by degenerating spermatids filled with cytoplasmic vacuoles (E), phagosomes containing shrunken electron-dense spermatids engulfed within Sertoli cells (F), and spermatogenic giant cells (G). Bars (A-D)=100 μm, (E)=10 μm, (F)=5 μm, (G) 10 μm.

FIG. 14 shows protein aggregates in YAC72 (−/−) mice. Ultrastructural analysis of the testes of YAC72 mice lacking endogenous htt revealed the occasional presence of abnormal aggregates of intracellular protein (arrows) within elongate spermatids (A), sertoli cells (B), and sperm tails (C). The composition of these protein aggregates is not entirely clear, but they resemble the ultrastructural appearance of huntingtin aggregates found in human HD brain tissue. Ectopic microtubule bundles (D) and manchettes (E) were also identified (asterisks). The bundle in E is in a spermatogonium. N=nucleus. Bars (A, B)=5 μm, (C,D)=1 μm, (E)=5 μm.

FIG. 15 presents immunocytochemical analysis of protein aggregates and actin distribution in sections from the testes of YAC72−/− mice. Abnormal protein aggregates within degenerating spermatogenic cells in the testes of YAC72−/− mice contain huntingtin (Figures A, phase, and B, immunofluorescence). In normal epithelium (Figures E, phase, and F, fluorescence), actin filaments in Sertoli cells are concentrated in unique adhesion plaques (ectoplasmic specializations) that occur at apical sites of attachment to spermatids (Apical) and at basal sites of attachment to neighbouring Sertoli cells (Basal). In YAC72−/− mice (FIGS. G-J), filament bundles (asterisks) in apical regions occur in areas not associated with spermatid heads, although filament bundles at basal sites occur in their normal position. Bars (A-D)=10 μm, (E-J)=50 μm.

FIG. 16 shows the effect of htt over-expression on body weight. Transgenic mice aged 5-8 months old that over-express wild-type htt (n=11) had a significantly increased total body mass compared to sex and age matched control mice (n=12). The average body weight was approximately 25% greater for the transgenic mice compared to wild-type. The increased body weight of the transgenic mice suggests that the wild-type htt is causing increased cellular survival or proliferation in general in these mice.

FIG. 17 shows the effect of transfecting NIH3T3 cells with a wild-type huntingtin gene or a known oncogene (ras). Cellular proliferation was assessed following transient transfections of NIH3T3 cells using a standardised colorometric assay of cell number. Transfection of a full-length htt construct or the oncogene ras each lead to a significant increase in cellular proliferation of approximately 3-fold compared to empty vector alone.

Table 1 demonstrates the outcome of crosses between YAC transgenic mice that are heterozygous for the Hdh null allele (huntingtin knockout mice). The total number of live-born offspring is given for each transgenic line. Note that the offspring have the expected 1:2:1 ratio indicating that there was no significant foetal loss and that the human transgene is capable of rescuing the Hdh nullizygous state.

### DETAILED DESCRIPTION OF THE INVENTION

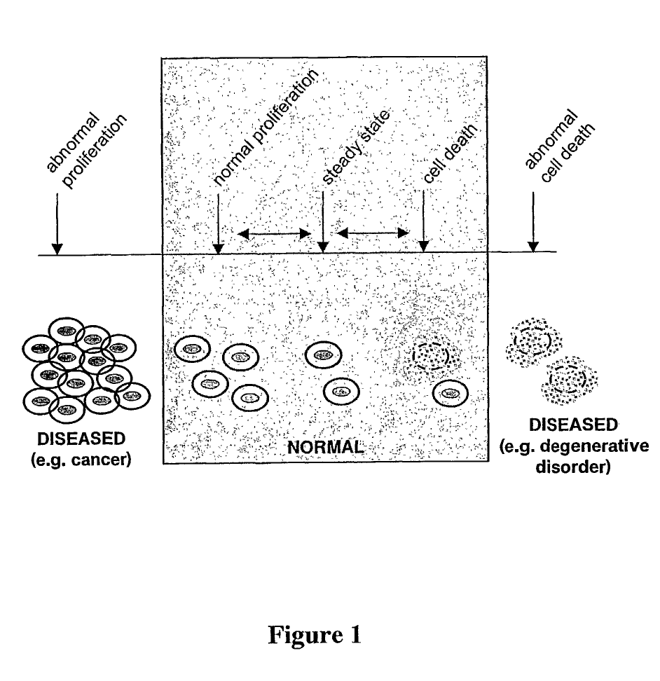

Cellular contents and cells themselves are constantly in a state of flux, known as turnover. Growth and proliferation of cells is intimately associated with cell death, for example, as a result of apoptosis or necrosis. The state of cells can vary between excess proliferation, normal proliferation, steady state, cell death and abnormal cell death (FIG. 1). The end result of disease or disorders oftentimes results from in an imbalance between the generative aspects of the lifecycle and the destructive aspects and the consequent abnormal proliferation of cells or abnormal cell death.

Thus, cancer can be seen to result from cellular growth being out of proportion from cellular death, resulting in rapid proliferation of cellular material. On the other hand, cellular death out of proportion from regeneration can result in the destruction of crucial areas of tissue, as observed in degenerative diseases such as, but not limited to, Parkinson's Disease, Amyotrophic Lateral Sclerosis, Alzheimer's disease or Huntington's Disease.

At the most general level, this invention provides huntingtin protein, biologically active fragments of the huntingtin protein, nucleic acid sequences encoding such proteins, and antagonists to Huntingtin protein, and their use to modulate cell survival. In particular, these components are used to either activate or attenuate the apoptotic pathway within cellular material, in order to facilitate the treatment of conditions where there is a dysregulation of cell death (e.g. apoptosis) or cellular proliferation. Thus, the therapeutic application pertains to diseases and disorders such as Huntington disease, neurodegenerative diseases, stroke, etc, where there is a need to attenuate apoptosis, in addition to diseases such as cancer where there is a need for decreased cellular proliferation.

Definitions

The techniques and procedures are generally performed according to conventional methods in the art and various general references (see generally, Sambrook et al. Molecular Cloning: A Laboratory Manual, 2d ed. (1989) Cold Spring Harbor Laboratory Press, Cold Spring Harbor, N.Y.) which are provided throughout this document. Standard techniques are used for chemical syntheses, chemical analyses, and biological assays.

As employed throughout the disclosure, the following terms, unless otherwise indicated, shall be understood to have the following meanings:

“Huntingtin” refers to the huntingtin protein, the mutant form of which is associated with Huntington disease.

“Naturally occurring” as used herein, as applied to an object, refers to the fact that an object can be found in nature. For example, a polypeptide or polynucleotide sequence that is present in an organism (including viruses) that can be isolated from a source in nature and which has not been intentionally modified by man in the laboratory is naturally occurring.

“Polypeptide fragment” refers to a polypeptide that has an amino-terminal and/or carboxy-terminal deletion, but where the remaining amino acid sequence is usually identical to the corresponding positions in the naturally occurring sequence deduced, for example, from a full-length cDNA sequence.

“C terminus” means the half of the wild-type huntingtin protein that includes the C-terminus. The term is used synonymously with C-terminal fragment or C-terminal domain.

“N terminus” means the half of the wild-type huntingtin protein that includes the N-terminus.

“Antagonist” as used herein means any molecule that is capable of interacting with huntingtin protein in such a way that it interferes with the normal pro-survival function of the protein. Examples of antagonists according to the present invention are antisense oligonucleotides and anti-huntingtin antibodies.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference).

The Use of Huntingtin Protein and Biologically Active Fragments Thereof

The present invention provides for the use of Huntingtin protein to attenuate cell death, thereby promoting cell survival, in the treatment of diseases or disorders wherein there is an imbalance between the generative and degenerative aspects of the cell, thus resulting in inappropriate cell death; such as, Alzheimer's disease, amytrophic lateral sclerosis, Huntingtin's disease, Parkinson's disease and retinal degeneration.

The huntingtin protein is expressed throughout the body, suggesting that taking advantage of the pro-survival properties of huntingtin, or biologically active fragments thereof, can be used in therapies where there is chronic excessive cell death (e.g. HD, Alzheimer's disease) or acute cell death (stroke and spinal cord injury).

The polyglutamine group of neurodegenerative diseases, including the spinocerebellar ataxias, HD, spinobulbar muscular atrophy (SBMA) and dentatorubralpallidoluysian atrophy (DRPLA), has been proposed to have similar molecular pathogeneses. For example, apoptotic cell death, caspase activation and protein deposition are common events in the degenerative processes of these disorders. Therefore, the pro-survival effect of huntingtin, or biologically active fragments thereof, can act as a general therapeutic for the entire group of polyglutamine diseases.

Additionally, aberrant aggregation is a common feature of many neurodegenerative diseases, including the polyglutamine diseases (intracellular inclusions), Alzheimer's disease (amyloid plaques) and Parkinson's disease (Lewy bodies). The formation of protein aggregates may be involved at some stage in disease pathogenesis. Huntingtin was shown to reduce aggregation of mutant huntingtin in vitro, suggesting a potential therapeutic role of the C-terminus in preventing “aggregation disease” in general.

Finally, recent work has shown the involvement of huntingtin in haematopoiesis (Metzler et al. (2000) Hum Mol Genet 9, 387-94), thus, huntingtin has potential therapeutic value in treatment of diseases with aberrant blood cell production and maturation.

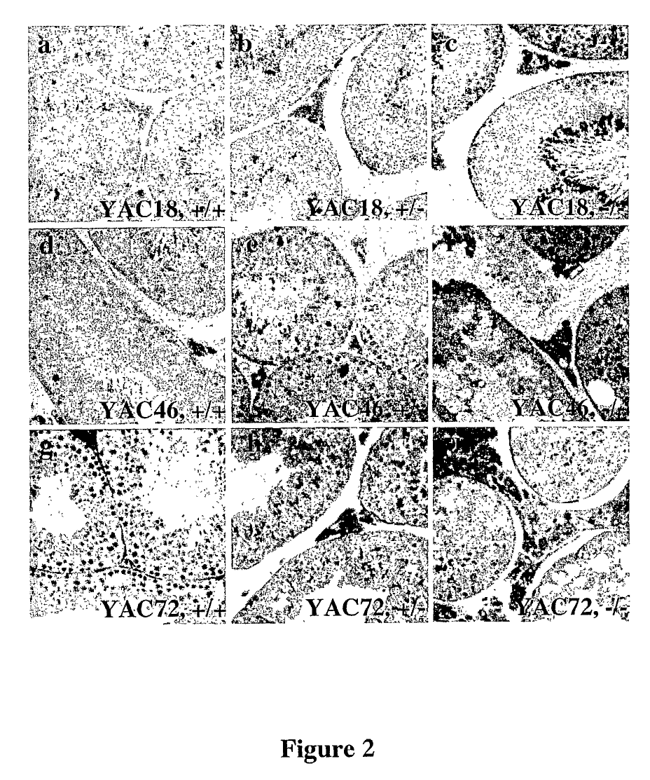

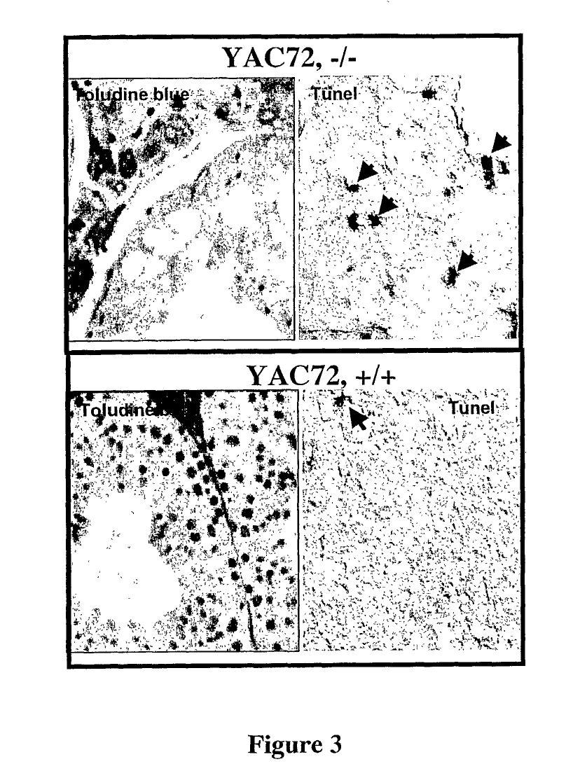

In the absence of wild-type huntingtin, the expression of mutant huntingtin in male HD mice results in infertility, testicular atrophy and apoptosis. Increasing the levels of endogenous wild-type huntingtin protects against the testicular phenotype. The results provided in FIGS. 2-4, 14 demonstrate that testicular cell death is CAG repeat size dependent and is modulated by the level of endogenous huntingtin. In particular, FIG. 2 clearly indicates that addition of exogenous normal huntingtin; with only 18 CAG repeats, does not cause cell death, whereas the addition of exogenous huntingtin with 46 or 72 CAG repeats results in increasing levels of cell death. In humans, 18 CAG repeats in the huntingtin protein does not result in disease formation, whereas both 46 and 72 repeats do cause HD in humans. Further, FIGS. 3, 4 and 14 show that increased apoptosis and protein aggregation can be attributed to the presence of huntingtin with 72 CAG repeats.

According to another embodiment of the present invention biologically active fragments of huntingtin protein can be used to attenuate the cellular pathway of apoptosis. These fragments can arise from any portion of the huntingtin protein. In one embodiment, such fragments arise from the C-terminus of the protein which further demonstrates that huntingtin is able to modulate apoptosis. It is not necessary, however, that the actual C-terminus be included in such fragments. Rather this portion of the protein provides a good source of candidate biologically active fragments that can then be further tested for activity.

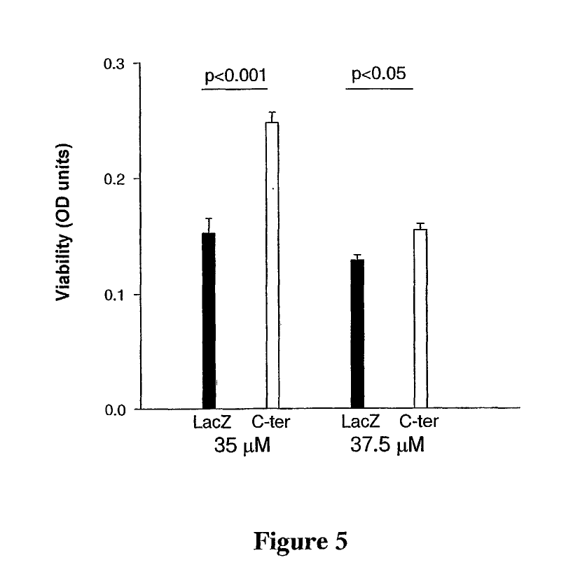

The data presented in FIGS. 5-8 demonstrates the effectiveness of biologically active fragments derived from the C-terminus of Huntingtin protein. Sequence analysis of the C-terminus has not identified any similarities to other proteins that would indicate its function. The observation of anti-apoptotic and anti-aggregation properties was a surprising result obtained from functional studies (described below) using a C-terminal fragment of the huntingtin protein.

One embodiment of the present invention is, therefore, the protection of cells from cell death as a result of the function of huntingtin protein and/or biologically active fragments derived from any portion of huntingtin.

Another embodiment is the reduction of aggregation as a result of the anti-aggregation activity, or function, of huntingtin protein and/or biologically active fragments derived from any portion of huntingtin.

Another embodiment of the present invention takes advantage of the activities of the biologically active fragments derived from huntingtin to protect mammals with HD from testicular degeneration and cell death. This has been demonstrated in male mice that are models for HD. Thus, the pro-survival activities of huntingtin, as outlined below, bring about the therapeutic effects that have been observed in male mice models for HD. This demonstrates that biologically active fragments derived from huntingtin act to regulate cell death, thereby improving cell survival and prognostic outcome of the condition being treated.

The pro-survival activity of biologically active fragments derived from huntingtin fits well with what is known about huntingtin, HIP-1 and caspase activation. It has been shown, in an in vitro HD model, that cleavage of huntingtin by caspase is necessary for cell death to occur. The resulting N-terminal polyglutamine-containing fragment of huntingtin has been shown to be toxic to cells. Furthermore, HIP-1 is a protein that interacts tightly with normal huntingtin but only weakly with mutant huntingtin. HIP-1 induces cell death by an apoptotic mechanism, and the amount of cell death is increased in the presence of mutant huntingtin.

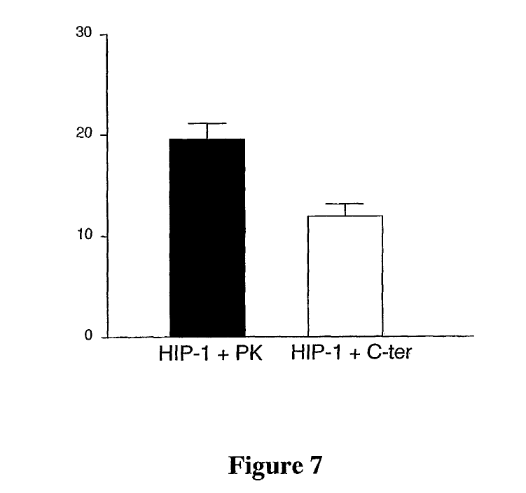

In one embodiment of the present invention there is provided a therapeutic means based on delivery of biologically active fragments derived from a domain of huntingtin, such as, but not limited to, the C-terminal domain. The ability of a fragment derived from the C-terminus to reduce HIP-1 mediated toxicity is demonstrated from neuronal cell culture studies in which the expression of huntingtin significantly reduced toxicity due to HIP-1 (FIG. 7). Therefore, if HD is partially or wholly due to HIP-1 toxicity, then delivery of peptides derived from huntingtin, or nucleic acid sequences encoding such protein, to susceptible cells will have a therapeutic benefit.

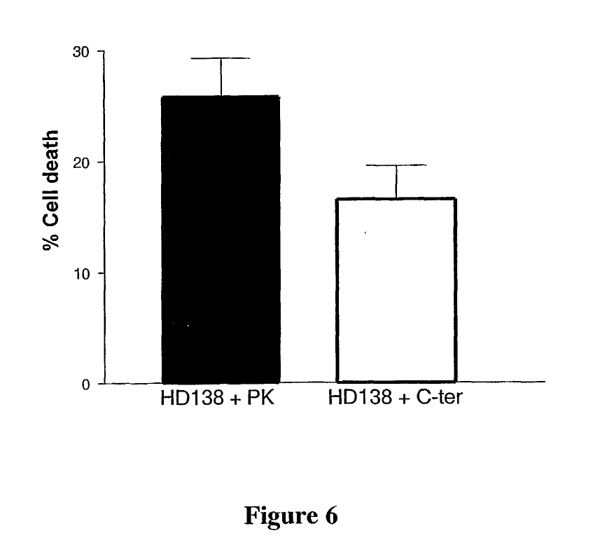

The ability of peptides derived from huntingtin to reduce polyglutamine-mediated toxicity has also been demonstrated in neuronal cell culture studies. For example, the expression of huntingtin was demonstrated to protect against cell death due to the presence of mutant huntingtin (FIG. 6).

An advantage of utilising biologically active fragments of huntingtin, such as, but not limited to, those arising from the C-terminus of huntingtin, as a therapeutic approach to the treatment of huntingtin and other polyglutamine disease, over alternative pharmaceutical-based therapies, lies in the specific targeting of part of the pathological pathway. This is an improvement over therapies aimed at the symptoms of the disease rather than at the cause.

Candidate fragments are selected from random fragments generated from the wild-type huntingtin. In one embodiment of the present invention the fragments are generated from the C-terminal domain of the wild-type huntingtin. Methods for generating the candidate polypeptide fragments are well known to workers skilled in the art and include enzymatic, chemical or mechanical cleavage of the native protein, expression of nucleic acids encoding such fragments, etc.

Assays to Determine the Pro-Survival Activity of a Candidate Molecule

Screening for the pro-survival effect of the candidate huntingtin protein fragment in vitro can be performed using cell lines transfected with the gene encoding the candidate fragment. The transfected cells are treated with a pro-apoptotic drug and one of several easily defined cellular markers of viability is measured. The markers used include, but are not limited to, morphological features of apoptosis, caspase activity and mitochondrial function. Additionally, cell lines can be created that stably express a biologically active fragment of huntingtin that can be used as a reagent for screening the effectiveness of the fragment in protecting against multiple pro-cell death stimuli.

The pro-survival effect of the huntingtin protein can be measured in vitro, for example, by transfecting cell lines with huntingtin, treating with a pro-cell death drug and assaying for several cellular markers. These markers include: (1) cell death, measured by cell morphology; (2) mitochondrial viability, measured by enzyme activity; and (3) aggregate formation, measured by immunofluorescence staining. In vivo effects of huntingtin in the transgenic mice can be assessed by: (1) observation of testicular cellular morphology by microscopy; and (2) DNA fragmentation using TUNEL staining.

1) Cell Death Assays.

In one example, human neuronal precursor cell line NT2 cells are transfected with huntingtin or control cDNAs using lipofectamine, according to the manufacturer's directions. Cell death can be quantified in NT2 cells by co-transfection of the expression constructs with a plasmid containing the LacZ gene at a 4:1 ratio, and the cells are stained for β-galactosidase activity at 24 hr post-transfection using standard procedures. The pro-survival effect of the candidate protein is assessed by incubating transfected cells with the pro-apoptotic drug tamoxifen at various concentrations. An apoptotic morphology is scored as blue-staining cells that are rounded up, blebbed and condensed, which are clearly distinguished from viable cells that are flat and have neurite-like extensions.

2) Mitochondrial Viability.

In one example, HEK 293T cells are seeded in 96-well plates and transfected with huntingtin or LacZ DNA using a standard calcium phosphate protocol as described (Hackam et al., 1998). Transfected cells in 96-well plates are treated at 48 hours post-transfection with a various concentrations of tamoxifen. Cell viability is measured by MTT assay at 24 hr post-transfection by incubating the cells for 2 hr in a 1:10 dilution of WST-1 reagent (Boehringer Mannheim) and release of formazan from mitochondria is quantified at 450 nm using an ELISA plate reader. Mock transfected, vector only and LacZ transfected cells serve as controls for transfection-related toxicity. One way ANOVA and Newman-Keuls test are used for statistical analysis. Statistical analyses of the cell death data in NT2 and HEK cells are performed using one-way ANOVA and Newman-Keuls post-comparison tests.

3) Aggregate Formation.

In one example, human embryonic kidney cells (HEK 293T) are transfected and processed for immunofluorescence by growing cells on glass coverslips and transfecting using a standard calcium phosphate protocol. At 48 h post-transfection, the cells are treated with tamoxifen to induce aggregate formation, then processed for immunofluorescence. The cells are fixed, permeabilised, then incubated with anti-huntingtin antibody. Secondary antibodies conjugated to a marker, such as FITC with the use of DAPI (4′,6′-diamindino-2-phenylindole) as a nuclear counter-stain. Appropriate control experiments are performed to determine the specificity of the antibodies, including secondary antibody only and mock transfected cells. The cells are viewed with microscope, digitally captured with a CCD camera and the images are colourised and overlapped. The proportion of cells with aggregates is presented as a percent of the total number of cells expressing huntingtin.

In vivo effects of huntingtin in the transgenic mice are assessed by, for example: (1) observation of breeding efficiency; (2) analysis of testicular cellular morphology by microscopy and DNA fragmentation using TUNEL staining.

b 1) Generation of Experimental Mice and Assessment of Breeding Efficiency.

In one example, heterozygous knockout (KO) mice are bred with YAC transgenic mice to generate a series of F1 generation mice that express the YAC transgene on a background that is heterozygous for endogenous huntingtin (one copy of the KO allele). These F1 generation mice are then bred to generate the experimental F2 mice with the following genotypes: YAC transgene positive or negative on a background of normal endogenous htt (+/+), half normal endogenous htt (+/−), and lacking endogenous htt (−/−). Genotyping is performed by standard PCR based techniques on genomic DNA from tail clippings prepared by phenol-chloroform extraction. Protein expression is determined by Western blot, using an anti-huntingtin antibody to probe the blot.

Mice of each genotype are set-up with FVB/NJ wild-type mates and allowed to remain in breeding cages for a minimum of 4 months with the number of pregnancies, litters, and pups recorded. Several breeding pairs are set-up per genotype and the results of a minimum of 20 months of combined breeding time tabulated per genotype.

2) Analysis of Testicular Morphology.

In one example, whole testes are removed from adult mice of each genotype and one testicle prepared for immunocytochemistry and TUNEL staining and one for semi-thin sections stained with toluidine blue and electron microscopy. For immunocytochemical and TUNEL analyses the testes are immersion-fixed overnight in paraformaldehyde, cyropreserved in sucrose solution, frozen, and cryostat sectioned. For immunocytochemistry, slide mounted sections are incubated in blocking solution for one hour and then in diluted primary antibody solution overnight. After serial washes in phosphate buffered saline (PBS), the sections are incubated in diluted secondary antibody, washed, and mounted under coverslips. TUNEL staining is performed on similar sections using techniques known in the art, for example by using the In situ cell death detection kit (Boehringer Mannheim), according to manufacturer's instructions. For semi-thin toluidine blue staining and EM analysis, testes are cut in 40 μm coronal sections using a vibratome collected in PBS, osmicated (1% OsO₄ in 0.1M cacodylate buffer), rinsed, and stained overnight in 2% aqueous uranyl acetate. All the sections used are dehydrated in ascending concentrations of ethanol and propylene oxide (1:1) and flat embedded in Eponate 12. Semi-thin sections (1.5 μm) are cut using a Leica Ultracut S ultramicrotome, counterstained with toluidine blue or cresyl violet, differentiated in 95% alcohol and coverslipped. Sections are visualised using a Nikon Microphot FXA equipped with a 60× oil immersion lens. Ultrathin sections (90 nm) are cut using a Leica Ultracut S ultramicrotome, counterstained with 5% aqueous uranyl acetate for 5 minutes followed by lead citrate for 5 minutes. Thin sections are examined using an electron microscope.

Antagonists of Huntingtin Protein

One embodiment of the present invention provides antagonists of huntingtin protein that decrease the pro-survival function of the protein and thereby reduce abnormal cell proliferation. A related embodiment of the present invention is the use of such antagonists in the treatment of diseases or disorders having a dysregulation of cellular proliferation. In these diseases and disorders cell proliferation is out of balance with cell death, which results in inappropriate cell growth. Cancer is one example of a disease characterised by excess cell proliferation.

A huntingtin antagonist according to the present invention can be an antibody that binds to the Huntingtin protein and effectively decreases or eliminates the pro-survival activity of said protein. Alternatively, an antagonist may be a biologically inactive form, or fragment, of huntingtin protein that interferes with the action of the wild-type protein; for example, dominant negative mutants of huntingtin protein.

In a related embodiment of the present invention the huntingtin antagonist is an antisense oligonucleotide that targets the cellular gene (or mRNA transcribed from the gene) that encodes huntingtin protein. The targeting process also includes determination of a site or sites within this gene for the antisense interaction to occur such that modulation of expression of the protein will result. Within the context of the present invention, an exemplary intragenic site is the region encompassing the translation initiation or termination codon of the open reading frame (ORF) of the gene. In the context of the present invention, “translation initiation codon” refers to the codon or codons that are used in vivo to initiate translation of an mRNA molecule transcribed from a gene encoding huntingtin protein, regardless of the sequence(s) of such codons.

The term “translation initiation codon region” refers to a portion of such an mRNA or gene that encompasses from about 25 to about 50 contiguous nucleotides in either direction (i.e., 5′ or 3′) from a translation initiation codon. Similarly, the term “translation termination codon region” refers to a portion of such an mRNA or gene that encompasses from about 25 to about 50 contiguous nucleotides in either direction (i.e., 5′ or 3′) from a translation termination codon. The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N⁷-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be target regions, and are particularly useful in situations where aberrant splicing is implicated in disease, or where an overproduction of a particular mRNA splice product is implicated in disease. Aberrant fusion junctions due to rearrangements or deletions are also targets. It has also been found that introns can also be effective target regions for antisense compounds targeted, for example, to DNA or pre-mRNA.

Once one or more target sites have been identified, oligonucleotides are chosen which are sufficiently complementary to the target, i.e., hybridise sufficiently well and with sufficient specificity, to give the desired effect of decreasing the pro-survival activity of huntingtin protein within the cell.

“Specifically hybridisable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridisable. An antisense compound is specifically hybridisable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

In the context of the present invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides can exhibit desirable properties such as, for example, enhanced cellular uptake, enhanced affinity for nucleic acid target and increased stability in the presence of nucleases. One embodiment of the present invention provides antisense oligonuceotides comprising from about 8 to about 50 nucleotides. In a related embodiment the antisense oligonucleotides comprise from about 15 to about 30 nucleotides.

An alternative embodiment of the present invention provides huntingtin antagonists that are small molecules which bind to huntingtin protein thereby interfering with the normal pro-survival function of the protein. Examples of small molecules include, but are not limited to, small peptides or peptide-like molecules. Similarly, the present invention provides small molecule antagonists that bind to a nucleic acid encoding wild-type huntingtin and interfere with expression of the protein, thereby reducing the pro-survival effect of the protein.

Assay to Determine the Antagonist Activity of a Candidate Molecule

One embodiment of the present invention provides an assay for use in screening candidate molecules for antagonist activity against huntingtin function. This assay can be used to screen candidate molecules for anti-proliferative functions, which can be used, for example, as anti-cancer therapeutics.

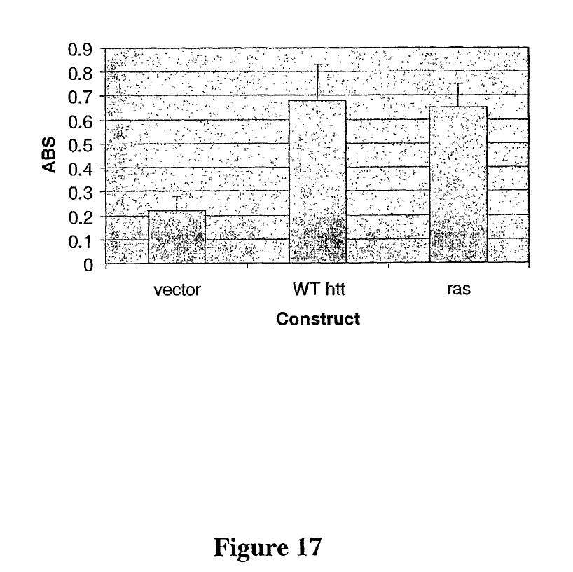

In one example of this assay NIH3T3 cells are first transfected with a wild-type huntingtin gene. The transfected cells show a significant increase in cellular proliferation (FIG. 17). The candidate molecule is added to the cell culture before, during or after proliferation of the transfected cells in vitro. This includes, but is not limited to, conditions in which the cells are cultured in a state of contact inhibition, in soft agar or in an animal. In one embodiment of the present invention the cells are cultured in a mouse in order to demonstrate cell proliferation or tumour growth in vivo. A control assay is performed simultaneously in the absence of the candidate molecule. The degree of proliferation in the presence of the candidate molecule is compared to the degree of proliferation in the control assay, where a reduced degree of proliferation in comparison to the control is indicative of an antagonist activity.

In a related embodiment the present invention provides screening kits comprising components required for performing the above anti-proliferation screening assay. Such a kit comprises NIH 3T3 cells transfected with wild-type huntingtin and instructions for use.

Preparation of Proteins and Polypeptides of the Present Invention

The proteins and polypeptide of the present invention, including wild-type huntingtin, biologically active fragments thereof and proteinaceous antagonists of wild-type huntingtin, can be prepared from cell extracts or through the use of recombinant techniques. In general, the proteins and polypeptides according to this invention can be produced by transformation (transfection, transduction, or infection) of a host cell with all or part of a DNA encoding such a protein or polypeptide in a suitable expression vehicle. Suitable expression vehicles include: plasmids, viral particles, and phage. For insect cells, baculovirus expression vectors are suitable. The entire expression vehicle, or a part thereof, can be integrated into the host cell genome. In some circumstances, it is desirable to employ an inducible expression vector, e.g., the LACSWITCH™ Inducible Expression System (Stratagene, LaJolla, Calif.).

Those skilled in the field of molecular biology will understand that any of a wide variety of expression systems can be used to provide the recombinant protein. The precise host cell used is not critical to the invention. The huntingtin protein can be produced in a prokaryotic host (e.g., E. coli or B. subtilis) or in a eukaryotic host (e.g., Saccharomyces or Pichia; mammalian cells, e.g., COS, NIH 3T3, CHO, BHK, 293, or HeLa cells; or insect cells). Proteins and polypeptides can also be produced using plant cells. For plant cells viral expression vectors (e.g., cauliflower mosaic virus and tobacco mosaic virus) and plasmid expression vectors (e.g., Ti plasmid) are suitable. The methods of transformation or transfection and the choice of expression vehicle will depend on the host system selected. Transformation and transfection methods are described, e.g., in Ausubel et al. (1994) Current Protocols in Molecular Biology, John Wiley & Sons, New York; and various expression vehicles may be chosen from those provided, e.g., in Cloning Vectors: A Laboratory Manual (Pouwels et al., 1985, Supp. 1987).

The host cells harbouring the expression vehicle can be cultured in conventional nutrient media adapted as need for activation of a chosen gene, repression of a chosen gene, selection of transformants, or amplification of a chosen gene.

Furthermore, the proteins and polypeptides of the present invention can be produced as fusion proteins. One use of such fusion proteins is to improve the purification or detection of the protein or polypeptide. For example, huntingtin or a fragment thereof, can be fused to an immunoglobulin Fc domain. Such a fusion protein can be readily purified using a protein A column.

Specific initiation signals may be required for efficient translation of cloned nucleic acid sequences. These signals include the ATG initiation codon and adjacent sequences. In cases where an entire wild-type huntingtin gene or cDNA, including its own initiation codon and adjacent sequences, is inserted into the appropriate expression vector, no additional translational control signals may be needed. In other cases, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al. (1987) Methods in Enzymol. 153, 516).

In addition, as would be readily appreciated by a worker skilled in the art, a host cell may be chosen which modulates the expression of the inserted sequences, or modifies and processes the gene product in a specific, desired fashion. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed.

Preparation of Compositions and Therapeutic Formulations

One embodiment of the present invention is the use of wild-type huntingtin, biologically active fragments of wild-type huntingtin, or antagonists of wild-type huntingtin protein in the preparation of pharmaceutical compositions, used for the treatment of conditions in which there is dysregulated cell death or cellular proliferation. Such pharmaceutical compositions comprise wild-type huntingtin, a biologically active fragment of wild-type huntingtin, a combination thereof, or one or more antagonists of wild-type huntingtin and a pharmaceutically acceptable diluent or excipient. A related embodiment is the compositions as above which additionally comprise a therapeutic compound, which may be chosen from the group comprising: antibiotics, anti-inflammatories, antidepressants, etc.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

Methods of Delivery for the Compositions and Formulations

The huntingtin protein, biologically active fragments thereof, or antagonists of huntingtin protein may also be employed in accordance with the present invention by expression of such proteins in vivo, which is often referred to as “gene therapy.”

Thus, for example, cells from a patient may be engineered with a polynucleotide (DNA or RNA) encoding a polypeptide ex vivo, with the engineered cells then being provided to a patient to be treated with the polypeptide. Such methods are well known in the art. For example, cells may be engineered by procedures known in the art by use of a retroviral particle containing RNA encoding huntingtin or a biologically active fragment thereof.

Similarly, cells may be engineered in vivo for expression of a polypeptide in vivo by, for example, procedures known in the art. As known in the art, a producer cell for producing a retroviral particle containing RNA encoding huntingtin, or a biologically active fragment thereof, may be administered to a patient for engineering cells in vivo and expression of the polypeptide in vivo. These and other methods for administering huntingtin, or a biologically active fragment thereof, by such method should be apparent to those skilled in the art from the teachings of the present invention. For example, the expression vehicle for engineering cells may be other than a retrovirus, for example, an adenovirus that may be used to engineer cells in vivo after combination with a suitable delivery vehicle.

In addition, when the antagonist of huntingtin comprises an antisense oligonucleotide, it is also contemplated that said oligonucleotide may be administered without additional carrier or delivery molecules. Injection of “naked” DNA is well-known in the art as an effective method of administering antisense therapy (for example, see Felgner et al., U.S. Pat. No. 5,580,589).

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions of the present invention may be employed in conjunction with other therapeutic compounds.

To gain a better understanding of the invention described herein, the following examples are set forth. It should be understood that these examples are for illustrative purposes only. Therefore, they should not limit the scope of this invention in any way.

### EXAMPLES

### Example 1

Determination of the In Vivo Effect of Reduced Normal Huntingtin

To determine the in Vivo effect of reduced expression of normal huntingtin, mice were bred to be transgenic for human huntingtin and to express various levels of wild-type mouse huntingtin. The breeding strategy allowed the generation of mice expressing huntingtin with different CAG repeat sizes, with or without co-expression of normal huntingtin.

Heterozygous knockout (KO) mice that had only one copy of the wild-type huntingtin allele were bred with YAC transgenic mice to generate a series of F1 generation mice that express the YAC transgene on a background that is heterozygous for endogenous huntingtin (“YAC rescued mice”). The YAC transgenes used in these studies contained 18, 46 or 72 CAG repeats. The F1 generation mice were then bred to generate the experimental F2 mice with the following genotypes: YAC transgene positive or negative on a background of normal endogenous htt (+/+), half normal endogenous htt (+/−), and lacking endogenous htt (−/−). Genotyping was performed by standard PCR based techniques on genomic DNA from tail clippings prepared by phenol-chloroform extraction (Ausubel et al. (1994) Current Protocols in Molecular Biology, John Wiley & Sons, New York). Protein expression was determined by Western blot in which 200 μg of total protein from homogenised testes was probed with the anti-huntingtin antibody (HD3).

The cellular effect of the huntingtin expression was determined by analysing the testicular morphology of YAC rescued huntingtin knockout mice. Spermatocyte degeneration, which was found to be CAG repeat size-dependent, was also shown to be modulated by the level of endogenous huntingtin (see FIG. 2).

Whole testes were removed from 8 month old adult male mice of each genotype. For semi-thin toluidine blue staining, testes were cut in 40 μm coronal sections using a vibratome, collected in PBS, osmicated (1% OsO₄ in 0.1M cacodylate buffer), rinsed, and stained overnight in 2% aqueous uranyl acetate. All the sections used were dehydrated in ascending concentrations of ethanol and propylene oxide (1:1) and flat embedded in Eponate 12 Semi-thin sections (1.5 μm) were cut using a Leica Ultracut S ultramicrotome, counterstained with Toludine Blue or Cresyl Violet, differentiated in 95% alcohol and coverslipped. Sections were visualised using a Nikon Microphot FXA equipped with a 60× oil immersion lens.

### Example 2

Quantitation of Increased Apoptosis with Mutant Huntingtin

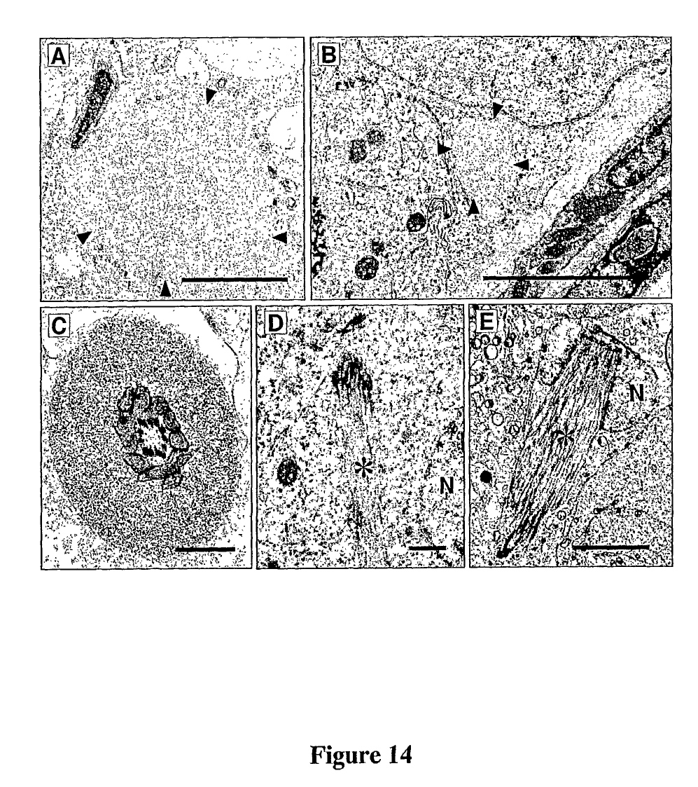

TUNEL staining was used to quantitate apoptosis in the YAC rescued transgenic mice. These experiments demonstrated increased apoptosis in the testes of YAC72 rescued huntingtin knockout mice, indicating that absence of endogenous huntingtin lead to cell death (see FIG. 14).

Whole testes were removed from adult mice of each genotype. For TUNEL analyses, the testes were immersion-fixed overnight in paraformaldehyde, cyropreserved in sucrose solution, frozen, and cryostat sectioned at 10 μm. For immunocytochemistry, slide mounted sections were incubated in blocking solution for one hour and then in diluted primary antibody solution overnight. After serial washes in phosphate buffered saline (PBS), the sections were incubated in diluted secondary antibody for two hours, washed, and mounted under coverslips. TUNEL labelling was performed using standard techniques on frozen sections of testes that were lightly immersion-fixed in 3% paraformaldehyde, using the In situ cell death detection kit (Boehringer Mannheim), according to the manufacturer's instructions.

### Example 3

Demonstration of Abnormal Protein Aggregation

Mutant polyglutamine containing proteins are known to aggregate into large amorphous protein clumps. These aggregates are considered to be markers for pathological changes in vivo, and their frequency correlates with cell death in culture. In the YAC72 transgenic mice, huntingtin aggregates were observed in the striata late in the pathological process (Hodgson et al. Neuron 1999, 23:181-92). Ultrastructural analysis using electron microscopy was used to determine the effect of endogenous huntingtin on the formation of aggregates. The testes of YAC72 mice lacking endogenous huntingtin contained abnormal aggregates within spermatids, sertoli cells and sperm tails (see FIG. 3).

For EM analysis, testes were cut in 40 μm coronal sections using a vibratome, collected in PBS, osmicated (1% OSO₄ in 0.1M cacodylate buffer), rinsed, and stained overnight in 2% aqueous uranyl acetate. All the sections used were dehydrated in ascending concentrations of ethanol and propylene oxide (1:1) and flat embedded in Ultrathin sections (90 nm) were cut using a Leica Ultracut S ultramicrotome, counterstained with 5% aqueous uranyl acetate for 5 minutes followed by lead citrate for 5 minutes. Thin sections were examined using a HITACHI H-7500 electron microscope.

### Example 4

Characterisation of Degeneration Phenotype

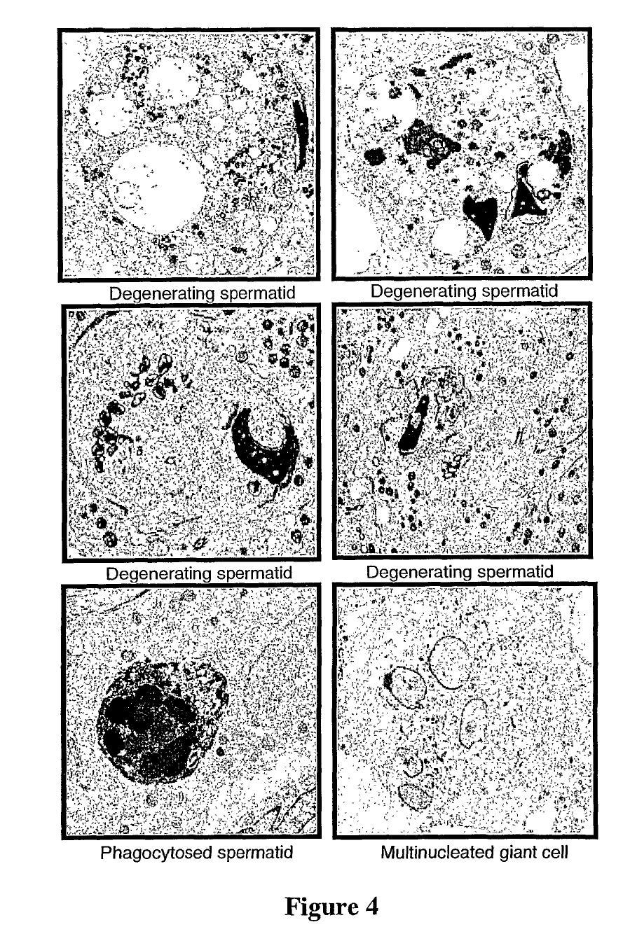

Additional electron microscopic analysis was used to characterise the degeneration phenotype in the YAC72 mice lacking endogenous huntingtin (see FIG. 4).

Ultrastructural analysis was performed as described in Example 3. The testes were shown to have elevated spermatid cell death, phagocytosis of degenerating cells and formation of multinucleated giant cells. These data demonstrate the role of endogenous huntingtin in protecting against mutant huntingtin-induced degeneration.

### Example 5

Demonstration of the Pro-Survival and Anti-Apoptotic Effect of Huntingtin

Tamoxifen-Induced Cell Death

Expression of the C-terminus of the huntingtin protein was shown to be protective against tamoxifen-induced cell death in transfected cell lines (see FIG. 5). Tamoxifen is a cell permeable compound that leads to caspase activation and cell death. The survival effect of huntingtin was measured in vitro by transfecting cell lines with huntingtin, treating with a pro-apoptotic drug and assaying for mitochondrial viability using the MTT assay. In the MTT assay, a reduction in mitochondrial viability is indicative of cell death.

For the viability assays, HEK 293T cells were seeded at a density of 5×10⁴ cells into 96-well plates and transfected with 0.1 μg huntingtin or LacZ DNA using a standard calcium phosphate protocol as described (Hackam et al. (1998) J Cell Biol. 141: 1097-105). Transfected cells in 96-well plates were treated at 48 hours post-transfection with a various concentrations of tamoxifen for 4 hr. Cell viability was measured, at 24 hr post-transfection, by incubating the cells for 2 hr in a 1:10 dilution of WST-1 reagent (Boehringer Mannheim) and release of formazan from mitochondria was quantified by the MTT assay at 450 nm using an ELISA plate reader (Dynatech Laboratories). Mock transfected, vector only and LacZ transfected cells served as controls for transfection-related toxicity. Statistical analyses of the cell death data were performed using one-way ANOVA and Newman-Keuls post-comparison tests.

Mutant Huntingtin-Induced Cell Death

An additional cell line was tested with a different cell death induction paradigm, to demonstrate that the C-terminus protection is not specific to tamoxifen-induced cell death. We show that the C-terminus of huntingtin confers protection against mutant huntingtin-induced toxicity in NT2 cells (see FIG. 6). Furthermore, this data supports the in vivo evidence that wild-type huntingtin is essential for protecting against mutant huntingtin in the YAC72 mice.

Human neuronal precursor cell line NT2 cells were co-transfected at 40% density with mutant huntingtin along with the C-terminus or control pyruvate kinase cDNAs, using lipofectamine (GibcoBRL), according to the manufacturer's directions. Cell death was quantified in NT2 cells by co-transfection of the expression constructs with a plasmid containing the LacZ gene at a 4:1 ratio, and the cells were stained for β-galactosidase activity at 24 hr post-transfection using standard procedures. The survival effect of the huntingtin protein was assessed by incubating transfected cells with the pro-apoptotic drug tamoxifen for 4 hrs at various concentrations. An apoptotic morphology was scored as blue-staining cells that were rounded up, blebbed and condensed, which were clearly distinguished from viable cells that were flat and had neurite-like extensions. The cell death data was analysed for statistical significance using one-way ANOVA and Newman-Keuls post-comparison tests.

HIP-1-Induced Cell Death

The huntingtin interacting protein HIP-1 is a pro-apoptotic protein that rapidly induces cell death. HIP-1 induces cell death in a caspase-dependent manner, and toxicity is exacerbated in the presence of mutant huntingtin, suggesting that HIP-1 may be involved in HD pathogenesis. We used HIP-1-induced toxicity to assess whether the C-terminus of huntingtin confers protection against a toxic protein-mediated cell death, in addition to its effect on tamoxifen-induced death (see FIG. 7).

NT2 cells were co-transfected with mutant HIP-1 and the C-terminus, or HIP-1 and the control pyruvate kinase cDNA, using lipofectamine (GibcoBRL). Cell death was measured by morphological changes using co-transfection of a plasmid containing the LacZ gene, as described above. We demonstrated that expression of the C-terminus in NT2 cells reduced cell death induced by HIP-1. These data indicate that the protective activity of the C-terminus may have a functional role in HIP-1-mediated cell death in vivo.

### Example 6

Reduction of Mutant Huntingtin Aggregation by C-Terminus of Huntingtin

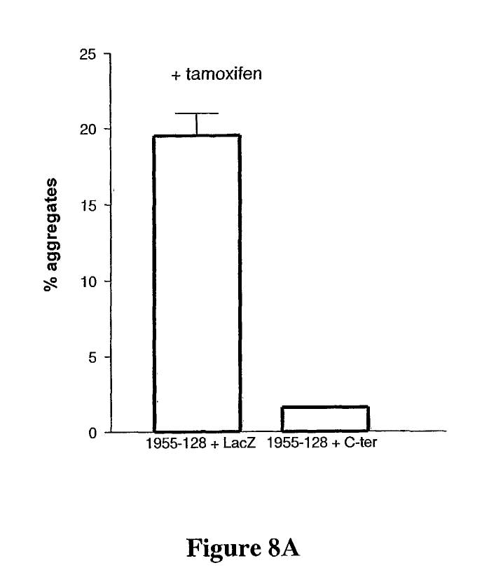

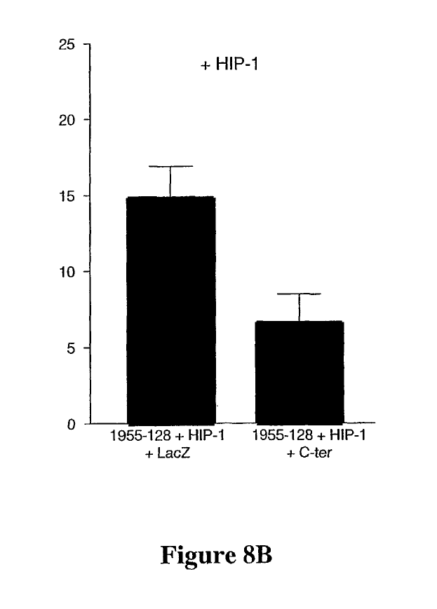

Mutant huntingtin protein forms aggregates in HD brains, transgenic mice and in cell culture. We have previously shown that truncated and full-length huntingtin containing 128 CAG repeats readily forms aggregates in HEK 293T cells when the cells are exposed to apoptotic stress by tamoxifen (Hackam et al. (1998) J Cell Biol. 141:1097-105). Therefore, huntingtin aggregates can be used as an additional marker of cell viability. To determine whether the C-terminus of huntingtin has an effect on this pathological marker of HD, we cotransfected the C-terminus with mutant huntingtin and induced aggregate formation with tamoxifen or HIP-1 expression (see FIG. 8).

Human embryonic kidney cells (HEK 293T) were transfected with truncated mutant huntingtin with 128 CAG repeats and processed for immunofluorescence by growing cells on glass coverslips and transfecting at 30% confluency using a standard calcium phosphate protocol. At 48 h post-transfection, the cells were treated with 35 μM tamoxifen (Sigma) for 1 hour to induce aggregate formation, then processed for immunofluorescence. The cells were fixed in 4% paraformaldehyde/PBS, permeabilised in 0.5% Triton X-100/PBS for 5 min, then incubated with anti-huntingtin antibody MAB2166 (Chemicon) (1:2500 dilution) diluted in 0.4% BSA/PBS. Secondary antibodies conjugated to FITC (Jackson Laboratories) were used at 1:600-1:800 dilutions, and DAPI (4′,6′-diamindino-2-phenylindole, Sigma) was used as a nuclear counter-stain. Appropriate control experiments were performed to determine the specificity of the antibodies, including secondary antibody only and mock transfected cells. The cells were viewed with a Zeiss (Axioscope) microscope, digitally captured with a CCD camera (Princeton Instrument Inc.) and the images were colourised and overlapped using the Eclipse (Empix Imaging Inc.) software program. The proportion of cells with aggregates is presented as a percent of the total number of cells expressing huntingtin. These data indicate that the C-terminus of huntingtin protein is able to reduce mutant huntingtin protein aggregate formation in transfected cells.

### Example 7

Offspring from Crosses of YAC Transgenic Mice

In Table 1 shows the offspring generated by crosses between YAC transgenic mice that are heterozygous for the huntingtin null allele (huntingtin knockout mice). These data demonstrate that there was no observed perinatal loss, and that the human transgene is capable of rescuing the huntingtin nullizygous state, indicating that the transgene has normal huntingtin developmental expression and function.

Mice of each genotype were set-up with FVB/NJ wild-type mates and allowed to remain in breeding cages for a minimum of 4 months with the number of pregnancies, litters, and pups recorded. Several breeding pairs were set-up per genotype and the results of a minimum of 20 months of combined breeding time tabulated per genotype.

### Example 8

Demonstration that Over-Expression of Full-Length Wild-Type Huntingtin in Mice Confers Protection Against Excitotoxic Neurodegeneration

Yeast artificial chromosome (YAC) transgenic mice were generated that over-express wild-type human huntingtin (line 212) at 2-3 times the levels of endogenous htt in wild-type mice (FVB/NJ) (Hodgson et al.(1996) Hum. Mol. Genet. 5, 1875-1885). Fifteen of these transgenic and 23 control FVB/NJ mice 4-8 months of age each received a single intraperitoneal injection of 25 mg/kg kainic acid (KA) or an equal volume of vehicle (PBS). Intraperitoneal injections of kainic acid injections cause prolonged seizures in mice, and each mouse was observed continuously for two hours following injection. The occurrence, severity, and duration of seizures was recorded.

One week following KA injection, brains were removed from one group of the mice and immediately frozen in isopentene on dry ice. Serial 30 μm coronal cryostat sections were cut through the entire hippocampus. After every fifth section, two sections were removed for quantitative analysis and fixed in 3% paraformaldehyde for 30 minutes in preparation for histochemical staining. Degenerating neurons were identified in hippocampal sections by Fluoro-Jade™ histochemistry (Histo-Chem inc.), silver staining (FD Neurotechnologies) and by TUNEL™ labeling (Boehringer Mannheim). Fluoro-Jade™ is a fluorescent stain that labels degenerating neurons in fixed brain sections. The total number of degenerated hippocampal neurons labeled with Fluoro-Jade™ was recorded from the CA1, CA3, and total hippocampus regions of each section selected for quantitative analysis. Slide-mounted sections were viewed with a Zeiss (Axiovert) fluorescent microscope, digital photomicrographs were captured with a cooled CCD camera (Princeton), and degenerating hippocampal neurons manually counted in a blinded fashion. To assess the role of caspase activation in this process, the hippocampus and cerebellum were removed from a second group of animals 8 hours following KA-induced seizures. Caspase activity was measured in homogenized hippocampal and cerebellar samples using the fluorogenic substrate acetylated DEVD aminofluorocoumarin. DEVD-ase activity was standardized to protein content as determined by standard Lowry analysis.

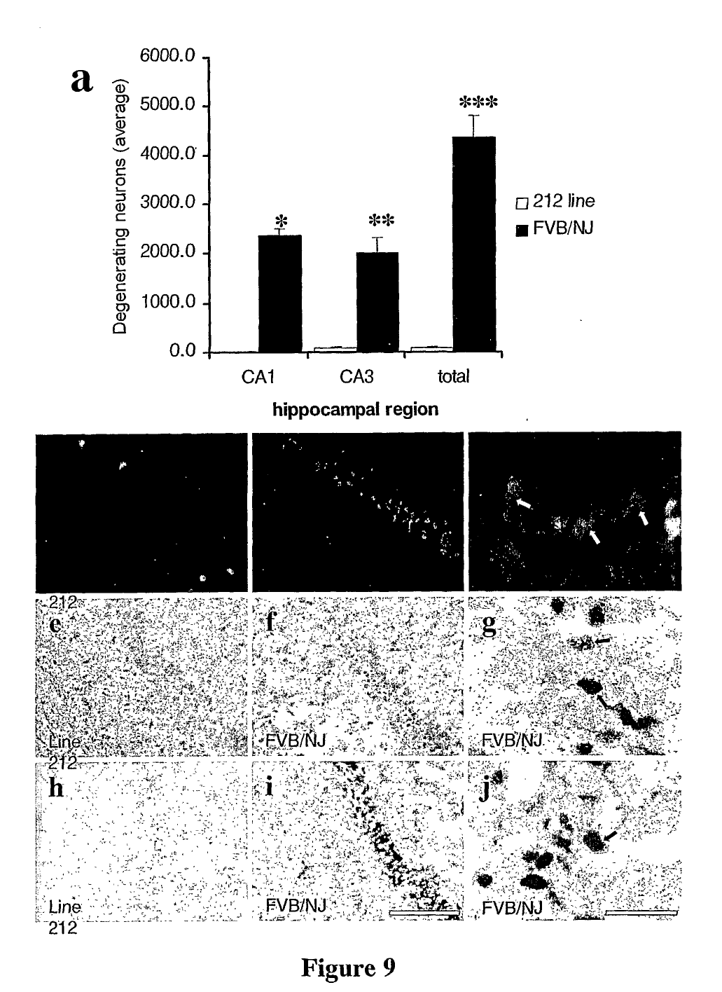

Following KA-induced seizures YAC transgenic mice expressing 2-3 times the endogenous levels of wild-type huntingtin averaged approximately 50-fold less (81 vs. 4362, *** p<0.0001) degenerating hippocampal neurons than control animals (FIG. 9a). Fluoro-jade brightly labeled the soma and large processes of degenerating hippocampal neurons within CA1 and CA3 of KA-treated transgenic and wild-type mice (FIG. 9b, 9c and 9d). Degenerating neurons were predominantly restricted to the CA1 and CA3 regions of hippocampus. KA-induced neurodegeneration was significantly reduced in both the CA1 (3 vs. 2360, * P<0.000001) and CA3 regions (79 vs. 2003, ** P<0.0003) of transgenic relative to control mice. Intraperitoneal injection of 25 mg/kg of kainic acid was sufficient to cause prolonged seizures in all the mice in this study irrespective of genotype. No seizure activity or neurodegeneration was observed in any mouse following injection of PBS.

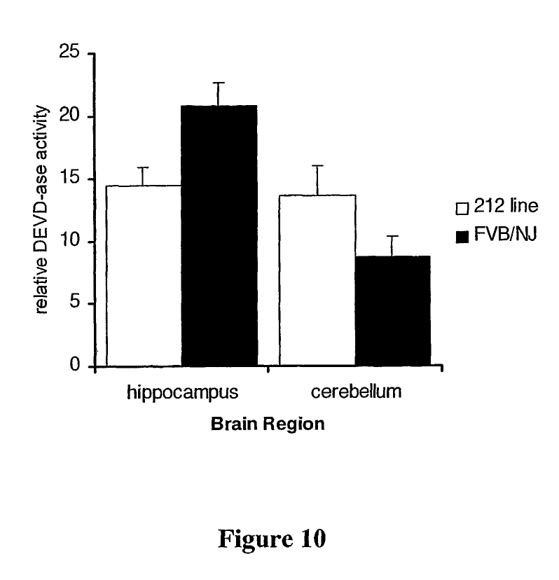

Argyrophyllic labelling of neuronal soma was dramatically reduced in adjacent silver-stained hippocampal sections from transgenic (FIG. 9e) compared to wild-type mice (FIG. 9f. and 9g.) following KA-induced seizures, confirming the results of Fluoro-Jade™ staining using this well-established marker for neurodegeneration. TUNEL™ staining (FIG. 9h.) labelled few apoptotic hippocampal neurons following KA-induced seizures in brains from transgenic mice, but many TUNEL™ labelled neurons were identified in wild-type brains (FIG. 9i and 9j). Significantly less hippocampal caspase activation was evident in transgenic mice compared to wild-type mice following KA-induced seizures (FIG. 10, 14.5 +/−1.49 vs. 20.8 +/−1.77, * P=0.02) in parallel to the observed effects of wild-type huntingtin on neurodegeneration. No significant difference was found in cerebellar caspase activation for transgenic vs. wild-type (FIG. 10, 13.66 +/−2.32 and 8.68 +/−1.67). Degeneration does not occur in the cerebellum following KA-induced seizures.

Transgenic mice expressing 2-3 times endogenous levels of wild-type huntingtin were resistant to apoptotic neurodegeneration, having approximately 50-fold less kainic acid-induced hippocampal neurodegeneration than littermate controls. Caspase activity levels within the hippocampus were increased following KA-induced seizures, and there was less DEVD-ase activation in the presence of increased levels of wild-type huntingtin. These data demonstrate a significant anti-apoptotic role for wild-type huntingtin in neurons of the central nervous system. This anti-apoptotic effect of wild-type huntingtin acts upstream of caspase activation. The DEVD-ase fluorogenic assay captures the enzymatic activity of caspase-2, -3, and -7, and does not identify the specific caspases activated in KA-induced seizures. Modulation of caspase-dependent pathways by wild-type huntingtin may alter the sensitivity of neurons to excitotoxic stress.

These results demonstrate that there is a relationship between normal huntingtin function and neuronal susceptibility to excitotoxicity. Increasing wild-type huntingtin expression levels either by drugs or using gene therapy has a therapeutic benefit as an anti-apoptotic agent for HD, and other neurodegenerative disorders.

### Example 9

Demonstration that Wild-Type Huntingtin Reduces the Cellular Toxicity of Mutant Huntingtin In Vivo

The mutation in Huntington disease (HD) is the expansion of a CAG trinucleotide repeat in the first exon of the HD gene (Huntington Disease Collaborative Research Group, 1993). Alleles containing expansions of greater than 35 CAG repeats are associated with the clinical phenotype of HD, with an earlier age of onset occurring with higher CAG repeat sizes (Andrew et al. (1993) Nat. Genet. 4, 398-403). The mutation in the HD gene produces a protein, huntingtin (htt), with an expanded polyglutamine tract. Proteolytic cleavage of huntingtin, possibly by caspases, produces N-terminal huntingtin fragments containing the expanded polyglutamine tract (Goldberg et al. (1996) Nat. Genet. 13, 442-449; Wellington et al. (1998) J. Biol. Chem. 273, 9158-9167; Wellington et al. (2000) J. Biol. Chem. 275, 19831-19838). N-terminal fragments of mutant expanded huntingtin have altered cellular interactions (Li et al. (1995) Nat. Genet. 25, 385-389; Burke et al. (1996) Nat. Med. 2, 347-349; Bao et al. (1996) Proc. Nat. Acad. Sci. USA 93, 5037-5042; Wanker et al. (1996) Hum. Mol. Genet. 6, 487-495; Kalchman et al. (1996) J. Biol. Chem. 271, 19385-19394; Kalchman et al. (1997) Nat. Genet. 16, 44-53), nuclear localisation (Davies et al. (1997) Cell 90, 537-548; DiFiglia et al. (1997) Science 277, 1990-1993; Becher et al. (1998) Neurobiol. Dis. 4, 387-397; Hackam et al. (1998) Cell. Biol. 141, 1097-1105; Schilling et al. (1999) Hum. Mol. Genet. 8, 397-407; Hodgson et al. (1999) Neuron 23, 181-192; Gutekunst et al. (1999) J. Neurosci. 19, 2522-2534; Wheeler et al. (2000) Hum. Mol. Genet. 9, 503-513; Li et al. (2000) Nat. Genet. 25, 385-389), and are directly toxic to neuronal cells in a variety of in vitro model systems (Martindale et al. (1998) Nat. Genet. 18, 150-154; Sandou et al. (1998) Cell 95, 55-66; Hackam et al. (1998) Cell. Biol. 141, 1097-1105). These huntingtin fragments are also prone to intracellular aggregation and inclusion formation (Hackam et al. (1998) Cell. Biol; 141, 1097-1105; Martindale et al. (1998) Nat. Genet. 18, 150-154; Cooper et al. (1998) Hum. Mol Genet. 7, 783-790), although the relevance of huntingtin aggregation to the pathogenesis of HD remains unclear (reviewed in Sisoda (1998) Cell 95,1-4). The expansion of polyglutamine residues in htt has been proposed to result in a novel toxic gain of function of the mutant protein (MacDonald and Gusella (1996) Curr. Opin. Neurobiol. 6, 638-643), but alterations in normal endogenous huntingtin levels and loss of htt function may also play a role in the pathogenesis of HD.

Huntingtin is a large protein of uncertain function that is ubiquitously expressed in many tissues of the body, but which has the highest levels in brain and testis (Sharp and Ross, (1996) Neurobiol. Dis. 3, 3-15). Mice homozygous for targeted disruption of Hdh (−/−), the murine homologue of the HD gene, die at embryonic day 7.5 (Nasir et al. (1995) Cell 81, 811-823; Duyao et al. (1995) Science 269, 407-410; Zeitlin et al. (1995) Nat. Genet. 11, 155-162). Mice with decreased levels of htt following targeted insertion of a neo construct into the Hdh gene have aberrant brain development and perinatal lethality (White et al. (1997) Nat. Genet. 17, 404-410). Mice heterozygous for targeted disruption of the Hdh gene (+/−) express half the normal levels of endogenous htt and develop neuronal degeneration in the basal ganglia in adulthood (O'Kusky et al. (1999) Brain. Res. 818, 468-479). Together, these results suggest that htt may normally be involved in both embryogenesis and in neuronal survival in the adult.

Recently, it was demonstrated that wild-type huntingtin has anti-apoptotic properties in vitro (Rigamonti et al. (2000) J. Neurosci. 20, 3705-3713). To assess the ability of wild-type huntingtin to modulate the toxicity of mutant huntingtin in vivo, a transgenic mouse model was developed in which the phenotypic effects of varying endogenous huntingtin levels on mice expressing transgenic huntingtin can be assessed.

Yeast artificial chromosome (YAC) mice were produced that are transgenic for the entire genomic region of the human HD gene, including all its regulatory sequences (Hodgson et al. (1999) Neuron 23, 181-192). Human huntingtin is expressed appropriately during development in YAC transgenic mice as demonstrated by the ability of the human transgene to rescue the embryonic lethality of Hdh nullizygous mice (−/−) (Hodgson et al. (1996) Hum. Mol. Genet. 5, 1875-1885). Appropriate control of tissue specificity was confirmed by the identical expression patterns of endogenous and human transgenic huntingtin by Western blot analysis and subcellular localization studies (Hodgson et al. (1996) Hum. Mol. Genet. 5, 1875-1885; Hodgson et al. (1999) Neuron 23, 181-192).

YAC transgenic mice have been generated that express human htt with 18 polyglutamines (YAC18) corresponding to a CAG repeat length observed in unaffected persons, 46 polyglutamines (YAC46) corresponding to a CAG repeat length observed in adult-onset HD patients, and 72 polyglutamines (YAC72) corresponding to a repeat length causing juvenile-onset HD (Hodgson et al. (1999) Neuron 23, 181-192). These mice express similar levels of transgenic human huntingtin differing only in polyglutamine expansion length. YAC18 mice have no observable phenotype up to 24 months of age, indicating that human huntingtin with a polyglutamine tract of normal length is not pathogenic in mice. However, mice transgenic for mutant huntingtin with an expanded polyglutamine develop a progressive phenotype characterized by behavioral, cellular and neuropathologic abnormalities similar to those observed in HD (Hodgson et al. (1999) Neuron 23, 181-192).

When crossed to the Hdh nullizygous background, YAC transgenic mice survive normally into adulthood (designated YAC −/−). These crosses provide a system in which one can examine the role of wild-type huntingtin in modulating the cellular toxicity of mutant huntingtin in vivo. In this study we demonstrate that mutant human huntingtin causes increased apoptotic cell death in the testes of transgenic mice expressing no endogenous htt. This pro-apoptotic effect of mutant huntingtin can be completely inhibited by increased levels of murine wild-type huntingtin, providing the first in vivo evidence that wild-type huntingtin can reduce the toxicity of mutant huntingtin.

Generation of Experimental Mice

YAC transgenic mice (FVB/NJ strain) from lines 29 (YAC18), 668 (YAC46), and 2511 (YAC72) were bred with mice heterozygous for targeted disruption of the endogenous mouse Hdh gene (C57BL6 strain) to produce F1 generation hybrid mice. F1 hybrid mice positive for the YAC transgene and heterozygous for the Hdh gene were then bred to produce litters of F2 experimental mice as previously described (Hodgson et al. (1996) Hum. Mol. Genet. 5, 1875-1885). All the F2 offspring of these matings were genotyped and used to generate experimental data. We also bred selected F2 mice to examine mating behavior, breeding success rates and to obtain post-coital sperm counts.

Genotyping was performed by standard PCR based techniques on genomic DNA from tail clippings prepared by phenol-chloroform extraction (Hodgson et al. (1996) Hum. Mol. Genet. 5, 1875-1885). Protein expression was determined by Western blot in which 200 ug of total protein from homogenized testes was loaded onto a low-bis acrylamide gel, run at 100V for 2 hours, and 200V for 3 hours before being transfereed to PVDF membranes. Blots were probed with anti-huntingtin antibody (HD3 @ 1/1000, Gutekunst et al. (1999) J. Neurosci. 19, 2522-2534) and detected using ECL (Amersham).

Fertility, Mating Behavior, and Sperm Analysis

To assess breeding success, adult male mice of each genotype were placed in breeding cages with single FVB/NJ female mice for at least four months (minimum of three male mice tested per genotype). The total duration of time spent in breeding cages and the total number of live-born litters was recorded for each mouse. Failure to produce any litters after a cumulative duration of breeding of at least 4 months was considered to represent male infertility. New wild-type females with previous successful breeding experience were placed in breeding cages of infertile males to control for any contribution of the female partner. YAC72−/− (mice expressing human htt with 72 polyglutamines and no endogenous murine htt), YAC72+/− (mice expressing human htt with 72 polyglutamines on a background of half the normal levels of endogenous murine htt), and YAC72+/+ (mice expressing human htt with 72 polyglutamines on a background of normal levels of endogenous murine htt) male mice were placed in breeding cages with pseudo-pregnant FVB/NJ females to assess male sexual behavior and post-coital sperm counts. These wild-type female mice were injected with 0.1 ml of pregnant mare serum (Sigma) 48 hours prior to stimulation with 10 IU of hcg (Sigma) and placement in breeding cages with the transgenic males. Mounting behavior of male mice was scored for 2-3 hours following placement in breeding cage and plug formation was determined by manual inspection of the female mice the following morning.

Post-coital sperm counts were determined from the extracted uterus of all female mice who had evidence of plug formation following breeding with male transgenic mice. Following breeding the plugged female mice were anaesthetized, the uterus and oviducts removed in toto and gently opened in a sterile 12 well tissue culture plate. 0.5 ml of sterile saline was used to flush the uterus, the resultant solution was collected, and examined microscopically for presence of sperm. To obtain quantitative sperm counts, the testes and epidydimi of YAC72+/+, YAC72+/−, and YAC72−/− mice (n=6 mice per genotype) were removed and immediately weighed. The tissues were then sectioned and placed in tubes containing 0.5 ml of sterile saline. Total numbers of morphologically normal sperm were manually counted using a brightline hemacytometer (Hausser) for three samples from each tissue. The hemacytometer counts for each tissue were averaged, and the total counts per ml were calculated. Results are expressed as average sperm count per ml +/+ SEM and significance was determined by the Students t-test.

Histological and Ultrastructural Analysis

Testes were removed from anesthetized animals and immediately placed in fixative (1.5% paraformaldehyde, 1.5% glutaraldehyde, 0.1 M Na cacodylate, pH 7.3). The capsules were nicked with a scalpel and then the organs left to fix for approximately 1 hour. The testes were cut into small pieces and fixed for an additional hour. The pieces were washed with buffer, post-fixed in buffered 1% OSO₄ on ice for 1 hour, washed with dH₂O, and stained en bloc in 1% aqueous uranyl acetate. The samples were washed with dH₂O, dehydrated through a graded concentration series of ethyl alcohols, and then embedded in JEMBED™ 812 (J. B. EM Services Inc., Point-Claire, Quebec).

For histological analysis, thick sections (1 μm) were stained with toluidine blue and evaluated on a Zeiss Axiophot microscope. For ultrastructural analysis, thin sections were cut on an ultramicrotome, stained with lead citrate and uranyl acetate, and then viewed and photographed on a Philips 300 electron microscope operated at 60 kV.

TUNEL™ labelling (Boehringer Mannheim) was performed using standard techniques on frozen sections of testes that were lightly immersion fixed in 3 % paraformaldehyde. Following removal of the testes, mice were injected with heparin and transcardially perfused with 3% paraformaldehyde and 0.15% glutaraldehyde in phosphate buffer (pH 7.4). Brains were then removed and post-fixed in 3% paraformaldehyde overnight.

Immunocytochemistry

Testes were excised from anesthetized animals, the capsules cut open with a scalpel, and the organs immersion fixed (3% paraformaldehyde, 150 mM NaCl, 5 mM KCl, 3.2 mM Na₂HPO₄, 0.8 mM KH₂PO₄, pH 7.3) for one to two hours. Following fixation, the testes were washed three times (10 minutes each wash) with PBS (150 mM NaCl, 5 mM KCl, 3.2 mM Na₂HPO₄, 0.8 mM KH₂PO₄, pH 7.3) and then frozen in OCT compound and sectioned on a cryostat. Sections (10 μm thick) were collected on polylysine-coated slides and then the slides were immediately plunged into cold acetone (−20° C.) for 5 minutes. Following this, the slides were air-dried.

Sections were re-hydrated for 30 minutes in TPBS (PBS, 0.05% Tween-20, 0.1% BSA) containing 5% normal goat serum (NGS), and then incubated for 1 hour at 37° C. with primary antiserum diluted 1:100 (HD3) with TPBS containing 1% NGS. Sections were washed (three times 10 minutes each wash) with TPBS, and then incubated for 1 hour at 37° C. with secondary antibody (goat anti-rabbit conjugated to Texas red) diluted 1:100 in TPBS. Sections again were washed with TPBS, mounted in Vetashield™ (Vector Laboratories, Burlingame, Calif.), and viewed on a Zeiss Axiophot microscope fitted with the appropriate fluorescence filter sets. Controls consisted of replacing the primary antibody with the same concentration of normal rat IgG (control for primary antibody), replacing the primary antibody with buffer alone (control for secondary antibody), and replacing the primary and secondary antibodies with buffer alone (control for autofluorescence).

Rescue of the Hdh−/−Lethal Phenotype by YAC Transgenes Expressing Mutant Human Huntingtin

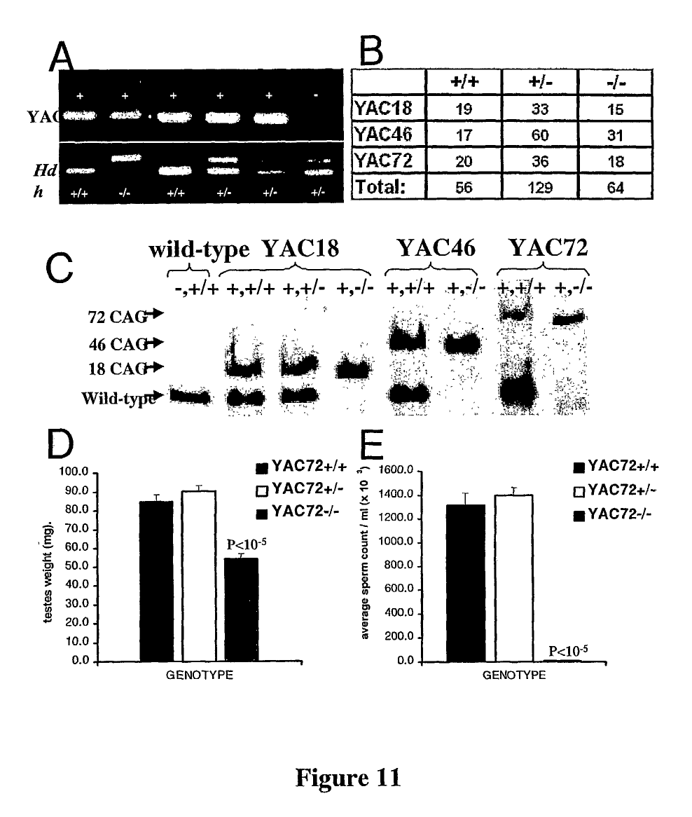

We generated yeast artificial chromosome (YAC) transgenic mice expressing normal (YAC18) or mutant (YAC46 or YAC72) human huntingtin in the absence of endogenous mouse htt (Hdh−/−). FIG. 11a demonstrates the genotype of several offspring from a cross between two mice heterozygous for targeted disruption of the Hdh gene, one of which also carried the YAC72 transgene (YAC72+/−). Mice with targeted disruption of both alleles of the Hdh gene can be rescued from the embryonic lethal phenotype by the YAC transgene expressing mutant huntingtin with 72 CAG repeats (YAC72−/− mice). In this litter, mice were generated with the YAC72 transgene and either 100% of the normal level of endogenous htt (YAC72+/+), 50% of the normal level of endogenous htt (YAC72+/−), or complete absence of endogenous htt (YAC72−/−). A mouse lacking the YAC72 transgene but heterozygous for endogenous huntingtin is also shown (FIG. 11A, −,+/−). No Hdh−/− mice were generated in the absence of the YAC transgene, consistent with the previous finding that Hdh nullizygous mice are not viable (Nasir et al. (1995) Cell 81, 811-823; Duyao et al. (1995) Science 269, 407-410; Zeitlin et al. (1995) Nat. Genet. 11, 155-162). The F2 offspring of our experimental breedings had the expected 1:2:1 ratio of genotypes for all of the YAC transgenes examined (FIG. 11B), demonstrating that both the normal (YAC18) and mutant (YAC46 and YAC72) human HD transgenes compensated for the lack of endogenous murine htt in Hdh−/− mice equally.

Huntingtin Expression Levels in YAC Transgene Rescued Hdh−/− Mice

Levels of the transgenic and wild-type huntingtin protein for YAC18 (line 29), YAC46 (line 668), and YAC72 (line 2511) Hdh−/− mice were measured by Western blot using an antibody recognizing both human and mouse htt (Hodgson et al. (1996) Hum. Mol. Genet. 5, 1875-1885). The human transgenic protein is expressed at similar levels in the YAC18, YAC46, and the YAC72 mice used in these experiments (FIG. 11C). This result was replicated in three different Western blots, and densiometric quantification of the transgenic protein level from these blots revealed a 1:1 ratio of transgenic versus endogenous htt levels in these mice. Transgenic protein levels from YAC18 mice averaged 102% of wild-type htt, YAC46 averaged 99% of wild-type htt, and YAC72 averaged 100% of wild-type htt. These 3 lines of YAC transgenic mice were selected for these experiments because they expressed identical levels of transgenic protein, differing only in the length of the CAG repeat.

Mice Expressing Mutant Huntingtin are Infertile

Expression of mutant (YAC46 and YAC72), but not wild-type (YAC18), transgenic huntingtin in the absence of endogenous htt leads to a novel phenotype that was initially identified by the observation that male Hdh −/− mice were unable to breed. We attempted to breed YAC46−/− and YAC72−/− males with wild-type females for extended periods with no success (no offspring were generated). Female littermates with the same genotype (YAC46−/− and YAC72−/−) had normal fertility when bred with wild-type mice. Male YAC72−/− mice had normal secondary sexual characteristics and displayed identical sexual behavior (mounting) and libido as YAC72+/− and YAC72+/+ mice when placed in cages with female wild-type mice. Similar plug formation rates were obtained for males of all three genotypes. Plugs were recovered in 8 of 12 breeding trials for YAC72−/− mice, 7 of 12 breeding trials for YAC72+/− mice and 8 of 16 breeding trials of YAC72+/+ mice. In sharp contrast to both YAC72+/+ and YAC72+/− mice, no post-coital sperm was ever recovered from plugged females following mating with YAC72−/− mice. These results suggested that a defect in spermatogenesis and not breeding behavior was responsible for the observed lack of fertility of male YAC72−/− mice.

Decreased Fertility in Mice Expressing Mutant Huntingtin is a Result of Decreased Sperm Production

To assess spermatogenesis directly in YAC72−/− mice, we performed sperm counts and examined the testes of these mice. YAC72−/− mice had significantly decreased epidydimal sperm counts compared to YAC72+/+ (11×10³/ml±2.9 vs. 13×10⁵/ml±100; p<0.00001) or YAC72+/−(11×10³/ml±2.9 versus 14×10⁵/ml ±65; p<0.00001) littermates at 4 months of age (FIG. 11D).

Testicular Atrophy and Spermatid Degeneration in Mice Expressing Mutant Huntingtin

YAC72−/− mice had significant testicular atrophy compared to YAC72+/+ (average testes weight, 54.0 mg±2.9 vs. 84.7 mg±3.6.9; p<0.00001) and YAC72+/− (average testes weight, 54.0 mg±2.9 vs. 90.1 mg±3.0; p<0.00001) littermates at 4 months of age (FIG. 11 E).

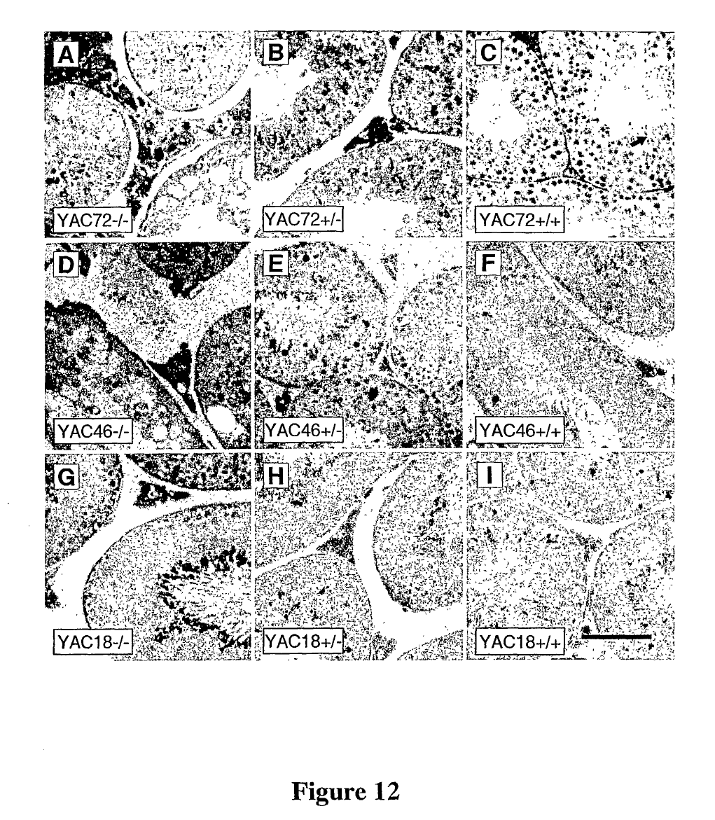

Histological examination of sections from the testes of adult mice expressing expanded mutant htt (YAC46−/− or YAC72−/−) stained with toluidine blue revealed massive disruption of spermatogenesis in the seminiferous tubules. The seminiferous tubules from these mice were full of large vacuoles and dying cells (FIG. 12A and D), but the spermatagonial stem cells and Sertoli cells close to the basement lamina appeared relatively normal in appearance and number. Depletion, but not absence, of cells at later stages of spermatogenesis (spermatocytes and spermatids) was evident in all layers of degenerating tubules. Degenerating cells at various stages of development were identified, suggesting that the spermatogenic defect caused by mutant htt is not limited to a single stage of development or due to defective maturation of spermatocytes. The normal stratified organization of cells within these seminiferous tubules was completely disrupted. Rarely, late spermatids were identified in the outer cell layer, but mature spermatazoa were not found in the lumen of these tubules, which were often filled with cellular debris (FIG. 12, A and D). Leydig cells appeared to be unaffected in the stromal interstitial tissue between degenerating tubules.

Cellular Degeneration in Mice Expressing Mutant Huntingtin can be Blocked by Expression of Endogenous htt and is CAG-length Dependent

This testicular degeneration was most striking in the testes of YAC72 −/− mice (FIG. 12A). Occasionally vacuolisation and cellular degeneration was seen in the testes from YAC72+/− mice expressing 50% of endogenous htt levels (FIG. 12B), but these mice were able to produce mature sperm (FIG. 11C). Increasing endogenous htt expression to 100% of normal levels in YAC72 mice completely rescued the degenerative testicular phenotype (FIG. 12C). Normal stratified organisation was restored to the seminiferous tubules, and no vacuolization or increased numbers of degenerating cells was present. Mature spermatozoa were found in the lumen of seminiferous tubules from YAC72+/+ mice (FIG. 12C, arrow) and these mice had normal fertility. These results suggest that the testicular cell death caused by the expression of polyglutamine expanded huntingtin in transgenic mice could be completely blocked by increasing the level of endogenous huntingtin.

The testicular cell death caused by the expression of polyglutamine expanded htt in transgenic mice (YAC46−/− and YAC72−/−) mice does not occur in mice expressing the same human transgene without the CAG repeat expansion (YAC18−/− mice, FIG. 12G). YAC18 mice lacking endogenous htt had normal morphology of the seminiferous tubules, no evidence of increased testicular cell death, and normal fertility. No significant effect was seen on YAC18 mice when levels of endogenous htt were increased to 50 or 100% of the normal huntingtin levels (FIG. 12H and I).

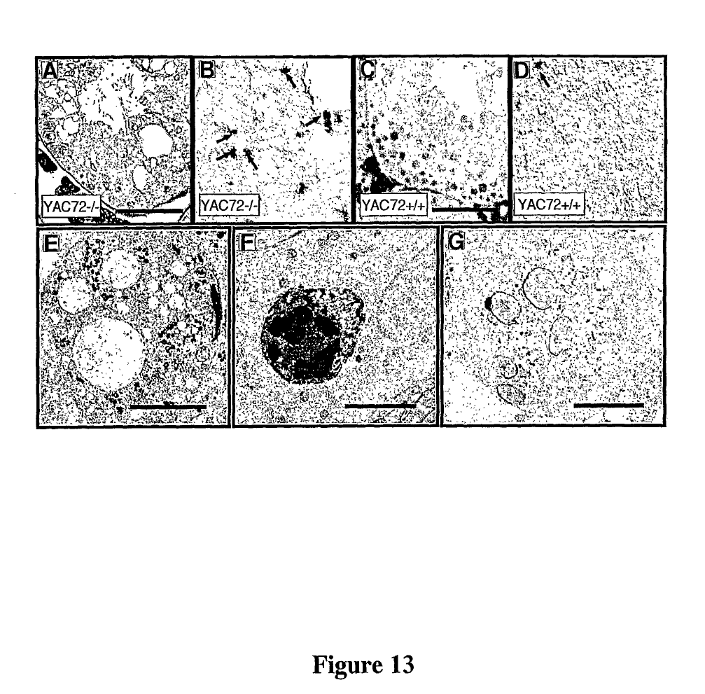

Histological examination of toluidine blue stained sections from the testes of adult mice expressing mutant htt revealed massive cellular death in multiple layers of the seminiferous tubules (FIG. 13A). TUNEL™ labelling (FIG. 13B, arrows) confirmed apoptotic nature of the widespread cell death in the testes of YAC72−/−. Despite the drastically reduced numbers of cells within seminiferous tubules from the YAC72−/− testes, the average number of TUNEL positive cells (3.2 per 20× field) was ˜10-fold higher in these sections than in sections from YAC72+/+ testes containing normal numbers of spermatogenic cells (0.3 per 20× field). No increased testicular cell death was observed in YAC72 mice expressing 100% of normal levels of endogenous huntingtin (YAC72+/+) either by toluidine blue (FIG. 13C) or by TUNEL™ labelling (FIG. 13D).

Ultrastructural Analysis (EM) of the Testes in YAC72 Mice Nullizygous for Endogenous Huntingtin (−/−) revealed large numbers of degenerating spermatids with diffuse cytoplasmic vacuolisation (FIG. 13E). Shrunken degenerating spermatids, with condensed nuclei and electron dense cytoplasm, were phagocytosed and degraded by Sertoli cells (FIG. 13F), suggestive of ongoing apoptosis and confirming the TUNEL™ findings. Multinucleated giant cells were found throughout the testes of YAC72−/− mice. These cells result from the opening of intercellular bridges between clones of spermatogenic cells. Importantly, no such degenerative phenotype was found by ultrastructural analysis of the testes of YAC18 mice (data not shown).

Abnormal Protein Aggregates Occur in the Testes of Mice Expressing Mutant Huntingtin

Ultrastructural analysis of the testes of YAC72−/− mice revealed the presence of occasional abnormal aggregates of intracellular protein (arrows) within spermatids (FIG. 14A), Sertoli cells (FIG. 14B), and sperm tails (FIG. 14C). These aggregates were rare, and found at an incidence of much less than one per high-powered field. No protein aggregates were identified in in YAC18−/− mice (data not shown). Ectopic microtubule bundles (FIG. 14D) and manchettes (FIG. 14E) were also occasionally identified (arrows) within spermatogonia and spermatids respectively. Also interesting is the observation that actin-containing adhesion plaques (ectoplasmic specializations) that occur in Sertoli cell cortical cytoplasm in regions of adhesion to spermatids often occurred in ectopic positions. Normally these structures occur in regions of attachment anly to spermatid heads. In YAC72−/− Sertoli cells, ectoplasmic specializations were observed to completely surround elongate spermatids that had re-acquired a circular form (data not shown).

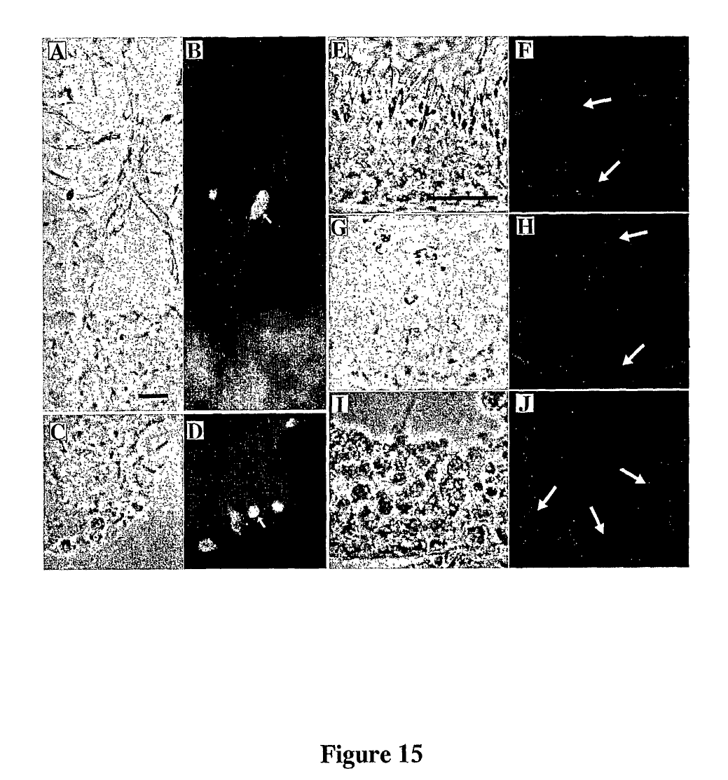

Immunocytochemical analysis of huntingtin localization in the testes of YAC72−/− mice revealed that protein aggregates within a small numbers of degenerating spermatids (FIG. 15, A and B) contain huntingtin. Similar huntingtin immunoreactivity was identified in aggregates within Sertoli cells adjacent to the basal lamina of degenerating seminiferous tubules (FIG. 15, C and D). Labelling of filamentous actin with fluorescent phallotoxin revealed altered localization of filaments within the testes of YAC72−/− mice (FIG. 15, I and J). In YAC72+/+ (Figure E and F), actin filaments were concentrated in Sertoli cell adhesion plaques (ectoplasmic specializations) found apically in association with spermatid heads and basally in association with junction complexes between neighbouring Sertoli cells. In YAC72−/− tissue (FIG. 15, I and J) actin filaments occur in linear arrays perpendicular to the tubule wall and in areas not directly related to spermatid heads (arrows with asterisks in FIG. 15, J).

The data herein provides evidence that wild-type huntingtin can significantly reduce the cellular toxicity of mutant huntingtin in vivo. Expression of human huntingtin with an expanded polyglutamine tract (46 and 72 polyglutamines) in the absence of wild-type htt results in male infertility, and massive apoptotic cell death in the testes in all phases of spermatogenesis. The cell death can be modulated by the expression of normal huntingtin. For example, mice expressing human huntingtin with 46 or 72 polyglutamines have no evidence for testicular atrophy or apoptosis in the testes when wild-type huntingtin is expressed from both Hdh alleles in the mouse. An intermediate phenotype is seen in these mice (YAC46 and 72) on the background of heterozygosity for targeted disruption in the mouse Hdh gene (YAC46 +/− or YAC72+/−). The severity of the testicular atrophy and apoptotic cell death is also modulated by the length of the polyglutamine repeat. YAC72 transgenic mice require higher levels of wild-type htt (+/+) than YAC46 transgenic mice (+/−) to prevent testicular degeneration. Abnormal protein aggregates, that contain htt, are occasionally found both in Sertoli cells and in spermatogenic cells in the testis of YAC72−/− mice. Also, structures containing cytoskeletal elements form ectopically in these mice suggesting that alterations in either the targeting of cytoskeletal elements to specific positions in the cells or of cytoskeletal function may be a mechanism promoting the massive apoptosis observed. These findings suggest that disruption of normal cytoskeletal organization may play a role in mediating the toxic effect of mutant huntingtin.

Both mutant and wild-type huntingtin proteins undergo cleavage (Wellington et al. (1999) J. Biol. Chem. 273, 9158-9167), and are recruited and sequestered into htt aggregates leaving less full-length wild-type huntingtin available to counteract pro-apoptotic stimuli (Martindale et al. (1998) Nat. Genet. 18,150-154). Polyglutamine expansion in one allele of the HD gene is associated with expression of half the cellular levels of wild-type huntingtin compared to normal neurons, which, together with huntingtin cleavage and sequestration of wild-type huntingtin in aggregates, may further decrease functional huntingtin levels. Mice heterozygous for targeted disruption of the Hdh gene express half the normal levels of wild-type huntingtin, and have previously been shown to develop neuronal degeneration in the basal ganglia (Nasir et al. (1995) Cell 81, 811-823; O'Kusky et al. (1999) Brain Res. 818, 468-479). One of the normal functions of huntingtin in the brain may be to protect cells against pro-apoptotic stimuli, and partial loss of this function may underlie some of the selective vulnerability of striatal neurons to cell death in HD. These data are in support of our previously stated hypothesis that loss of function of wild-type htt may also contribute to the pathogenesis of HD ((Nasir et al. (1995) Cell 81, 811-823).

Wild-type human huntingtin can protect hippocampal neurons from kainic acid-induced excitotoxicity. Furthermore, the very recent report that inactivation of Hdh expression in adult mice is associated with progressive apoptotic neurodegeneration provides further support for the anti-apoptotic role of wild-type huntingtin in the adult brain (Dragatsis et al. (2000) Nat. Genet. 26, 300-306).

A number of observations indicate that the ultimate outcome of huntingtin toxicity in the testis is the apoptotic loss of spermatogenic cells. The dramatic vacuolisation within the seminiferous tubules, TUNEL™ staining of spermatogenic cells, and the ectopic positioning of manchettes (microtubule structures associated with spermatid nucleii) in these cells are consistent with this conclusion. The presence of giant cells in the epithelium, the occurrence of large phagosomes containing spermatids in Sertoli cells and the obviously reduced numbers of spermatogenic cells in the epithelium demonstrated by our EM analysis all point to the conclusion that the primary testicular phenotype in YAC72−/− mice is apoptotic death of spermatogenic cells, particularly of spermatids.

Although morphological changes and cell loss are most dramatic in the spermatogenic cell population in YAC72−/− testes, Sertoli cells also express abnormal features. Two of these features are the presence of protein aggregates in the cytoplasm and the ectopic positioning of actin filament containing junction plaques normally found in regions adjacent to spermatid heads. Abnormal positioning of ectoplasmic specializations adjacent to spermatids cells may be a response to a primary defect in the associated spermatogenic cells. The presence of normally positioned junction plaques in basal regions of attachment to neighbouring Sertoli cells is consistent with this conclusion. These findings suggest that mutant huntingtin causes an intrinsic polyglutamine-mediated apoptotic cell death within spermatogenic cells, which can be blocked by wild-type huntingtin.

It has previously been shown that human huntingtin can compensate for the critical function of murine huntingtin during gastrulation by rescuing mice with targeted disruption on both Hdh alleles studies (Hodgson et al. (1996) Hum. Mol. Genet. 5, 1875-1885). These mice are rescued by both normal (YAC18) and mutant huntingtin (YAC46 and YAC72), clearly indicating that expansion of the polyglutamine does not disturb this important role of huntingtin in development. Here we have provided further evidence for cross-species functional complementarity of huntingtin. The presence of murine huntingtin can completely protect cells against the pro-apoptotic effects of mutant human huntingtin. Cross-species functional complementation between mouse and human huntingtin in development and in protection against apoptosis must reflect their high degree of sequence conservation with mouse and human huntingtin sharing complete identity of nucleotides at a level of 90% similarity in amino acid structure (Lin et al. (1994) Genomics 25, 707-715. The data provided herein demonstrates strong in vivo evidence that wild-type huntingtin can significantly modulate the apoptotic toxicity of mutant huntingtin, and we suggest that wild-type huntingtin may normally have an anti-apoptotic function. Mapping the critical region of huntingtin responsible for this function and investigation of the mechanism by which this critical region influences cell death pathways may identify novel therapeutic targets for HD and advance our understanding of huntingtin's normal role in the delicate balance between life and death in cells.

Role of Wild-type Huntingtin on Cell Proliferation and Tumor Formation in vivo.

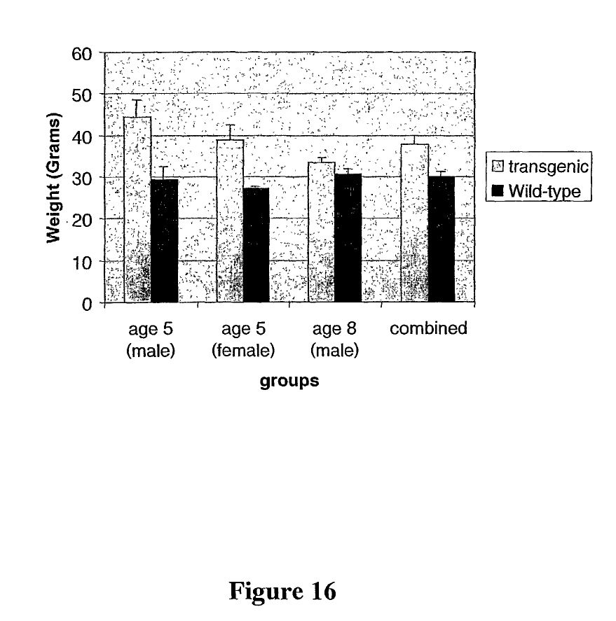

One of the roles of the pro-survival (e.g. anti-apoptotic) function of wild-type huntingtin may be in control of tissue mass and cellular proliferation. Increased levels of wild-type htt expression can lead to increased body tissue mass and may predispose mice to the development of tumors. The effect of over-expression of human wild-type huntingtin on the body weight of transgenic mice was compared to wild-type mice (FIG. 16). A significant increase in body weight was observed in transgenic mice compared to control mice, suggesting that increasing wild-type huntingtin function increases the number of cells in the body. Over production of cells in the body can have a variety of unwanted outcomes including but not limited to obesity or cancer. This effect could be a result of a decreased amount of naturally occurring apoptosis or increased cellular proliferation.

A retrospective analysis was performed to examine the total numbers of mice (identified by the animal colony health staff) that developed tumours in the colony of transgenic mice. The records of all mice diagnosed with tumours over a 6 month period were examined, in a group of mice over-expressing wild-type human huntingtin and their wild-type littermates. The vast majority of mice that developed tumours were found to be transgenic mice that over-expressed wild-type huntingtin (38/45 or 84.5 % of mice identified with tumours were transgenic). The average age of tumour development was younger in the transgenic group 13.8 months compared to 17.1 months for wild-type littermates. The majority of identified tumours in male mice were testicular tumours. Huntingtin is expressed at very high levels in the testes suggesting that this tissue may be at increased risk of tumour development when huntingtin levels are increased.

Role of Huntingtin on Cell Proliferation in vitro.

Huntingtin is required for the normal cellular proliferation of hematopoetic cells (Metzler et al. (2000) Hum. Mol. Genet. 9, 387-394). In these experiments embryonic stem cells that had decreased levels or complete absence of huntingtin were exposed to hematopoetic cytokines that stimulate cellular proliferation and found to have less response (less proliferation) than stem cells that had the normal levels of huntingtin. This early data suggests that huntingtin can have both anti-apoptotic and proliferative functions.

The ability of NIH3T3 cells to overcome contact inhibition when transfected with different gene constructs has been used for many years to assess oncogenic potential of certain genes and to identify potential oncogenes (genes that cause cancer). Using a standard screen of cellular proliferation in NIH3T3 cells, the effect that huntingtin has on cellular growth was examined and compared to the effect of known oncogenes (FIG. 17). NIH3T3 cells transfected with huntingtin constructs show a significant increase in cellular proliferation (5-6 fold increase in proliferation compared to control vector) that is similar to the effect of a well-described oncogene, ras.

The in vitro and in vivo data demonstrates that the anti-apoptotic function of huntingtin plays a role in controlling cellular proliferation and that alterations in this function can cause cancer or neurodegeneration depending on whether huntingtin function is increased or decreased respectively.

While the foregoing invention has been described in some detail for purposes of clarity and understanding, it will be appreciated by one skilled in the art from a reading of this disclosure that various changes in form and detail can be made without departing form the true scope of the invention and appended claims.

TABLE 1
Offspring of HD Knock-out Rescue Breedings

| | +/+ | +/− | −/− |
|---|---|---|---|
| YAC18 | 19 | 33 | 15 |
| YAC46 | 17 | 60 | 31 |
| YAC72 | 9 | 8 | 4 |
| Total: | 45 | 101 | 49 |

The embodiments of the invention in which an exclusive property or privilege is claimed are defined as follows:

1. A method of regulating cell survival comprising the step of altering levels of huntingtin function.

2. Use of a protein according to the method of claim 1, to modulate cell death or cell proliferation in a mammal having a condition characterised by a dysregulation of cell death or cell proliferation, wherein said protein is selected from the group consisting of huntingtin and a biologically active fragment of huntingtin protein.

3. Use of a nucleic acid encoding huntingtin, or a biologically active fragment thereof, according to the method of claim 1, to modulate cell death or cell proliferation in a mammal having a condition characterised by a dysregulation of cell death or cell proliferation.

4. Use of an antagonist of huntingtin according to the method of claim 1, to modulate cell proliferation in a mammal having a condition characterised by a dysregulation of cell proliferation.

5. The use according to claim 2 or 3, wherein said protein inhibits cell death.

6. The use according to any one of claim 2, 3 or 4, wherein said protein inhibits cell proliferation.

7. The use according to any one of claim 2, 3 or 5, wherein said condition is a neurodegenerative disease.

8. The use according to claim 7, wherein said neurodegenerative disease is Huntingtin's disease, any other polyglutamine disorder, Alzheimer's disease, amyotrophic lateral sclerosis or Parkinson's disease.

9. The use according to any one of claim 2, 3, 4 or 6, wherein said condition is cancer.

10. The use according to claim 9, wherein said cancer is a germ cell cancer.

11. The use according to claim 10, wherein said germ cell cancer is testicular cancer.

12. The use according to claim 4, wherein said antagonist is an antisense oligonucleotide.

13. The use according to claim 4, wherein said antagonist is an anti-huntingtin antibody.

14. The use according to claim 4, wherein said antagonist is a small molecule that binds to huntingtin or to a nucleic acid encoding huntingtin.

15. A nucleic acid encoding a biologically active fragment of huntingtin protein.

16. An antagonist of huntingtin.

17. The antagonist according to claim 16 which is an antisense oligonucleotide.

18. The antagonist according to claim 16 which is an anti-huntingtin antibody.

19. The antagonist according to claim 16 which is a small molecule that binds to huntingtin or to a nucleic acid encoding huntingtin.

20. A pharmaceutical composition comprising a pharmaceutically acceptable excipient and huntingtin, a biologically active fragment of huntingtin, or a combination thereof.

21. A pharmaceutical composition comprising a pharmaceutically acceptable excipient and a nucleic acid encoding huntingtin, a nucleic acid encoding a biologically active fragment of huntingtin, or a combination thereof.

22. A pharmaceutical composition comprising a pharmaceutically acceptable excipient and an antagonist of huntingtin.

23. The pharmaceutical composition according to claim 22 which is an antisense oligonucleotide.

24. The pharmaceutical composition according to claim 22 which is an anti-huntingtin antibody.

25. The pharmaceutical composition according to claim 22 which is a small molecule that binds to huntingtin or to a nucleic acid encoding huntingtin.

26. Use of huntingtin, a biologically active fragment of huntingtin, or a combination thereof for the preparation of a medicament for the treatment of conditions characterised by dysregulation of cell death or cell proliferation.

27. Use of a nucleic acid encoding huntingtin or a biologically active fragment thereof, for the preparation of a medicament for the treatment of conditions characterised by a dysregulation of cell death or cell proliferation.

28. Use of an antagonist of huntingtin for the preparation of a medicament for the treatment of conditions characterised by a dysregulation of cell proliferation.

29. The use according to claim 28, wherein said antagonist is an antisense oligonucleotide.

30. The use according to claim 28, wherein said antagonist is an anti-huntingtin antibody.

31. The use according to claim 28, wherein said antagonist is a small molecule that binds to huntingtin or to a nucleic acid encoding huntingtin.

32. An assay for screening for molecules having an anti-proliferative activity comprising the steps of:
(a) transfecting NIH3T3 cells with huntingin;
(b) culturing the transfected cells in the presence and absence of a candidate molecule;
(c) comparing proliferation of the transfected cells in the presence of the candidate molecule with proliferation of the transfected cells in the absence of the candidate molecule,
wherein an anti-proliferative activity is identified a decrease in the proliferation of the transfected cells in the presence of the candidate molecule in comparison to the proliferation of the transfected cells in the absence of the candidate molecule.

33. The assay according to claim 32, wherein in step (b) the transfected cells are cultured in a state of contact inhibition.

34. The assay according to claim 32, wherein in step (b) the transfected cells are cultured in soft agar.

35. The assay according to claim 32, wherein in step (b) the transfected cells are cultured in an animal.

36. The assay according to claim 35, wherein in said animal is a mouse.
